(19) United States
(12) Patent Application Publication
(10) Pub. No.: US 2002/0106655 A1
(43) Pub. Date: Aug. 8, 2002

(54) Human GPCR proteins

(76) Inventors: Olga Bandman, Mountain View, CA (US); Preeti G. Lal, Santa Clara, CA (US); Y. Tom Tang, San Jose, CA (US); Mariah R. Baughn, San Leandro, CA (US)

Correspondence Address: INCYTE GENOMICS, INC., 3160 Porter Drive, Palo Alto, CA, 94304, US

(21) Appl. No.: 09/895,686

(22) Filed: Jun. 28, 2001

Related U.S. Application Data
(63) Continuation-in-part of application No. 09/156,513, filed on Sep. 17, 1998, now abandoned.

(51) Int. Cl. C12Q 1/68; G01N 33/53; C07H 21/04; C12P 21/06; C07K 5/00; C07K 7/00; C07K 16/00; C07K 17/00; A61K 38/00

(52) U.S. Cl. 435/6; 536/23.5; 530/324; 435/7.1

(57) ABSTRACT

The invention provides human GPCR proteins and their encoding cDNAs. It also provides for the use of the cDNAs, proteins, and antibodies in the diagnosis, prognosis, treatment and evaluation of therapies for neoplastic disorders and immune response. The invention further provides vectors and host cells for the production of the protein and transgenic model systems.

This application is a continuation-in-part of U.S. Ser. No. 09/156,513, filed Sep. 17, 1998.

### FIELD OF THE INVENTION

This invention relates to a human GPCR proteins and their encoding cDNAs and to the use of these biomolecules in the diagnosis, prognosis, treatment and evaluation of therapies for neoplastic, neurological, and immune disorders.

### BACKGROUND OF THE INVENTION

Phylogenetic relationships among organisms have been demonstrated many times, and studies from a diversity of prokaryotic and eukaryotic organisms suggest a more or less gradual evolution of molecules, biochemical and physiological mechanisms, and metabolic pathways. Despite different evolutionary pressures, the proteins of nematode, fly, rat, and man have common chemical and structural features and generally perform the same cellular function. Comparisons of the nucleic acid and protein sequences from organisms where structure and/or function are known accelerate the investigation of human sequences and allow the development of model systems for testing diagnostic and therapeutic agents for human conditions, diseases, and disorders.

The term receptor describes proteins that specifically recognize other molecules. The category is broad and includes proteins with a variety of functions. Most receptors are cell surface proteins which bind extracellular ligand. The binding process leads to cellular activities including growth, differentiation, endocytosis, and immune response. Some receptors facilitate the transport of specific molecules across the endoplasmic reticulum or to a particular location in the cell.

G protein coupled receptors (GPCR) are a superfamily of integral membrane proteins which transduce extracellular signals. GPCRs include receptors for biogenic amines; lipid mediators of inflammation, peptide hormones, and sensory signal mediators. Activation of the GPCR by an extracellular ligand leads to intracellular conformational changes which enhance the binding affinity of a G protein, which is heterotrimeric and contains α β, and γ subunits, for GTP. Activation of the G protein by GTP leads to the interaction of the G protein a subunit with adenylate cyclase or another second messenger molecule generator. This interaction regulates the activity of adenylate cyclase in the production of a second messenger molecule, cAMP. cAMP, in turn, regulates phosphorylation and activation of other intracellular proteins. Alternatively, cellular levels of other second messenger molecules, such as cGMP or eicosinoids, may be upregulated or downregulated by the activity of GPCRs. GTPase deactivates the G protein a subunit by hydrolysis of GTP releasing the second messenger molecule generator so that the β,γ, and a subunits of the G protein can reassociate. Activity of a GPCR may also be regulated by phosphorylation of the intra- and extracellular domains or loops.

Visual excitation and the phototransmission of light signals is a signaling cascade in which GPCRs play an important role. The process begins in rod cells of the retina with the absorption of light by the photoreceptor rhodopsin, a GPCR composed of a 40-kDa protein, opsin, and a chromophore, 11-cis-retinal. The photoisomerization of the retinal chromophore causes a conformational change in the opsin GPCR and activation of the associated G-protein, transducin. This activation leads to the hydrolysis of cyclic-GMP and the closure of cyclic-GMP regulated, Ca²⁺-specific channels in the plasma membrane of the rod cell. The resultant membrane hyperpolarization generates a nerve signal. Recovery of the dark state of the rod cell involves the activation of guanylate cyclase leading to increased cyclic-GMP levels and the reopeni mng of the Ca²⁺-specific channels (Stryer (1991) J Biol Chem 266:10711-10714).

Glutamate receptors form a group of GPCRs that are important in neurotransmission. Glutamate is the major neurotransmitter in the CNS and is believed to have important roles in neuronal plasticity, cognition, memory, learning and some neurological disorders such as epilepsy, stroke, and neurodegeneration (Watson and Arkinstall (1994) The G-Protein Linked Receptor Facts Book, Academic Press, San Diego Calif., pp 130-132). These effects of glutamate are mediated by two distinct classes of receptors termed ionotropic and metabotropic. lonotropic receptors contain an intrinsic cation channel and mediate fast, excitatory actions of glutamate. Metabotropic receptors are modulatory, increasing the membrane excitability of neurons by inhibiting calcium dependent potassium conductances, and both inhibit and potentiate excitatory transmission of ionotropic receptors. Metabotropic receptors are classified into five subtypes based on agonist pharmacology and signal transduction pathways and are widely distributed in brain tissues.

The vasoactive intestinal polypeptide (VIP) family is a group of related polypeptides whose actions are also mediated by GPCRs. Key members of this family are VIP itself, secretin, and growth hormone releasing factor (GRF). VIP has a wide profile of physiological actions including relaxation of smooth muscles, stimulation or inhibition of secretion in various tissues, modulation of various immune cell activities, and various excitatory and inhibitory activities in the CNS. Secretin stimulates secretion of enzymes and ions in the pancreas and intestine and is also present in small amounts in the brain. GRF is an important neuroendocrine agent regulating synthesis and release of growth hormone from the anterior pituitary (Watson and Arkinstall supra, pp 278-283).

The structure of GPCRs is highly-conserved and consists of seven hydrophobic transmembrane (serpentine) regions, cysteine disulfide bridges between the second and third extracellular loops, an extracellular N-terminus, and a cytoplasmic C-terminus. Three extracellular loops alternate with three intracellular loops to link the seven transmembrane regions. The most conserved parts of these proteins are the transmembrane regions and the first two cytoplasmic loops. A conserved, acidic-Arg-aromatic residue triplet present in the second cytoplasmic loop may interact with the G-proteins. The consensus pattern of the G-protein coupled receptors signature (PS00237; SWISSPROT) is characteristic of most proteins belonging to this superfamily (Watson and Arkinstall supra, pp 2-6).

The discovery of new human GPCR proteins and their encoding cDNAs satisfies a need in the art by providing compositions which are useful in the diagnosis, prognosis, treatment and evaluation of therapies for neoplastic, neurological, and immune disorders.

### SUMMARY OF THE INVENTION

The present invention is based on the discovery of human GPCR proteins and their encoding cDNAs which are expressed in neoplastic, neurological, and immune disorders. The cDNAs, proteins and an antibody which specifically binds each protein are useful in the diagnosis, prognosis, treatment and evaluation of therapies for neoplastic, neurological, and immune disorders, particularly follicular carcinoma of the thyroid, leiomyoma of the uterus, pancreatic cancer, epilepsy, interstitial nephritis, and immune response as a complication of cancer.

The invention provides an isolated cDNA comprising a nucleic acid sequence encoding a protein having the amino acid sequence of SEQ ID NOs: 1-6. The invention also provides an isolated cDNA selected from a nucleic acid sequence of SEQ ID NOs:7-12, fragments of SEQ ID NOs:7-12 selected from SEQ ID NOs: 13-52, and variants of SEQ ID NOs:7-12 selected from SEQ ID NOs:53-74 and the complements of SEQ ID NOs:7-74. The invention additionally provides compositions, a substrate, and a probe comprising the cDNA or the complement of the cDNA. The invention further provides a vector comprising the cDNA, a host cell comprising the vector and a method for making a protein comprising culturing a host under conditions to produce the protein and recovering the protein from culture. The invention still further provides a transgenic cell line or organism comprising the vector containing the cDNA encoding a GPCR. The invention additionally provides a fragment, a variant, or the complement of a cDNA selected from SEQ ID NOs: 13-74. In one aspect, the invention provides a substrate containing at least one nucleotide sequence selected from SEQ ID NOs:7-74 or the complements thereof. In a second aspect, the invention provides a probe comprising a cDNA or the complement thereof which can be used in methods of detection, screening, and purification. In a further aspect, the probe is selected from a single-stranded RNA or DNA molecule, a peptide nucleic acid, a branched nucleic acid and the like.

The invention provides a method for using a cDNA to detect the differential expression of a nucleic acid in a sample comprising hybridizing a probe to the nucleic acids, thereby forming hybridization complexes and comparing hybridization complex formation with at least one standard, wherein the comparison confirms the differential expression of the cDNA in the sample. In one aspect, the method of detection further comprises amplifying the nucleic acids of the sample prior to hybridization. In another aspect, the method showing differential expression of the cDNA is used to diagnose infection, inflammation or cancer, particularly meningioma of the brain. In yet another aspect, the cDNA or a fragment or a variant or the complements thereof may comprise an element on an array.

The invention additionally provides a method for using a cDNA or a fragment or a variant or the complements thereof to screen a library or plurality of molecules or compounds to identify at least one ligand which specifically binds the cDNA, the method comprising combining the cDNA with the molecules or compounds under conditions allowing specific binding, and detecting specific binding to the cDNA , thereby identifying a ligand which specifically binds the cDNA. In one aspect, the molecules or compounds are selected from aptamers, DNA molecules, RNA molecules, peptide nucleic acids, artificial chromosome constructions, peptides, transcription factors, repressors, and regulatory molecules.

The invention provides a purified protein or a portion thereof selected from the group consisting of an amino acid sequence of SEQ ID NOs: 1-6, a variant of SEQ ID NOs: 1-6, an antigenic epitope of SEQ ID NOs: 1-6, and a biologically active portion of SEQ ID NOs: 1-6. The invention also provides a composition comprising the purified protein and a pharmaceutical carrier. The invention further provides a method of using a GPCR to treat a subject with infection, inflammation or cancer comprising administering to a patient in need of such treatment the composition containing the purified protein or a portion thereof. The invention still further provides a method for using a protein to screen a library or a plurality of molecules or compounds to identify at least one ligand, the method comprising combining the protein with the molecules or compounds under conditions to allow specific binding and detecting specific binding, thereby identifying a ligand which specifically binds the protein. In one aspect, the molecules or compounds are selected from DNA molecules, RNA molecules, peptide nucleic acids, peptides, proteins, mimetics, agonists, antagonists, antibodies, immunoglobulins, inhibitors, and drugs. In another aspect, the ligand is used to treat a subject with infection, inflammation and cancer, particularly meningioma of the brain.

The invention provides a method of using a protein to screen a subject sample for antibodies which specifically bind the protein comprising isolating antibodies from the subject sample, contacting the isolated antibodies with the protein under conditions that allow specific binding, dissociating the antibody from the bound-protein, and comparing the quantity of antibody with known standards, wherein the presence or quantity of antibody is diagnostic of infection, inflammation and cancer, particularly meningioma of the brain.

The invention also provides a method of using a protein to prepare and purify antibodies comprising immunizing a animal with the protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified antibodies.

The invention provides a purified antibody which binds specifically to a protein which is expressed in infection, inflammation or cancer. The invention also provides a method of using an antibody to diagnose infection, inflammation or cancer comprising combining the antibody comparing the quantity of bound antibody to known standards, thereby establishing the presence of infection, inflammation or cancer. The invention further provides a method of using an antibody to treat infection, inflammation and cancer comprising administering to a patient in need of such treatment a composition comprising the purified antibody and a pharmaceutical carrier.

The invention provides a method for inserting a heterologous marker gene into the genomic DNA of a mammal to disrupt the expression of the endogenous polynucleotide. The invention also provides a method for using a cDNA to produce a mammalian model system, the method comprising constructing a vector containing the cDNA of SEQ ID NOs:53-74, transforming the vector into an embryonic stem cell, selecting a transformed embryonic stem cell, microinjecting the transformed embryonic stem cell into a mammalian blastocyst, thereby forming a chimeric blastocyst, transferring the chimeric blastocyst into a pseudopregnant dam, wherein the dam gives birth to a chimeric offspring containing the cDNA in its germ line, and breeding the chimeric mammal to produce a homozygous, mammalian model system.

### BRIEF DESCRIPTION OF THE TABLE AND FIGURES

Table 1 characterizes the receptors of the invention. Column 1 contains the SEQ ID NO; column 2, the number of the amino acids in the sequence; column 3, potential phosphorylation sites; column 4, potential glycosylation sites; column 5, signature sequences (or motifs) derived using the analytical methods/databases described in column 7 or other public databases such as the GenBank rodent, mammalian, vertebrate, prokaryote, and eukaryote databases and SwissProt; and column 6, identification or classification of each GPCR.

FIG. 1A and 1B are a clustal alignment of the metabotropic glutamate receptors, SEQ ID NOs: 1 and 5 produced using the multiple alignment program of LASERGENE software (DNASTAR, Madison WI).

FIGS. 2A and 2B are a clustal alignment of the somatostatin and rhodopsin-like receptors, SEQ ID NOs:2-4 produced using the multiple alignment program of LASERGENE software (DNASTAR, Madison Wis.).

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“Array” refers to an ordered arrangement of at least two cDNAs or antibodies on a substrate. At least one of the cDNAs or antibodies represents a control or standard, and the other, a cDNA or antibody of diagnostic or therapeutic interest. The arrangement of two to about 40,000 cDNAs or of two to about 40,000 monoclonal or polyclonal antibodies on the substrate assures that the size and signal intensity of each labeled hybridization complex, formed between each cDNA and at least one nucleic acid, or antibody:protein complex, formed between each antibody and at least one protein to which the antibody specifically binds, is individually distinguishable. “GPCR protein” refers to a purified protein obtained from any mammalian species, including bovine, canine, murine, ovine, porcine, rodent, simian, and preferably the human species, and from any source, whether natural, synthetic, semi-synthetic, or recombinant.

A “complement” of a cDNA of the Sequence Listing refers to a nucleic acid molecule which is completely complementary to the cDNA over its full length and which will hybridize to the cDNA or an mRNA under conditions of maximal stringency. “cDNA” refers to an isolated polynucleotide, nucleic acid molecule, or any fragment or complement thereof. It may have originated recombinantly or synthetically, may be double-stranded or single-stranded, represents coding and noncoding 3′ or 5′ sequence, and generally lacks introns.

A “composition” refers to the polynucleotide and a labeling moiety , a purified protein and a pharmaceutical carrier, an antibody and a labeling moiety, and the like.

“Derivative” refers to a cDNA or a protein that has been subjected to a chemical modification. Derivatization of a cDNA can involve substitution of a nontraditional base such as queosine or of an analog such as hypoxanthine. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by presence, absence or at least two-fold change in the amount or abundance of a transcribed messenger RNA or translated protein in a sample.

“Disorder” refers to conditions, diseases or syndromes in which the cDNAs and receptors are specifically and differentially expressed. These include, but are not limited to, diagnosis, prognosis, treatment and evaluation of therapies for neoplastic, neurological, and immune disorders, particularly follicular carcinoma of the thyroid, leiomyoma of the uterus, pancreatic cancer, epilepsy, interstitial nephritis and immune response as a complication of cancer.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′base pairs with 3′-T-C-A-G-5′. Hybridization conditions, degree of complementarity and the use of nucleotide analogs affect the efficiency and stringency of hybridization reactions.

“Labeling moiety” refers to any visible or radioactive label than can be attached to or incorporated into a cDNA or protein. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which may be used in hybridization or amplification technologies or in regulation of replication, transcription or translation. Equivalent terms are amplimer, primer, and oligomer.

An “oligopeptide” is an amino acid sequence from about five residues to about 15 residues that is used as part of a fusion protein to produce an antibody.

“Portion” refers to any part of a protein used for any purpose; but especially, to an epitope for the screening of ligands or for the production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR).

“Purified” refers to any molecule or compound that is separated from its natural environment and is from about 60% free to about 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint, buccal cells, skin, or hair; and the like.

“Similarity” refers to the quantification (usually percentage) of nucleotide or residue matches between at least two sequences aligned using a standard algorithm such as Smith-Waterman alignment (Smith and Waterman (1981) J Mol Biol 147:195-197) or BLAST2 (Altschul et al. (1997) Nucleic Acids Res 25:3389-3402). BLAST2 may be used in a reproducible way to insert gaps in one of the sequences in order to optimize alignment and to achieve a more meaningful comparison between them. Particularly in proteins, similarity is greater than identity in that conservative substitutions (for example, valine for leucine or isoleucine) are counted in calculating the reported percentage. Substitutions which are considered to be conservative are well known in the art.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

THE INVENTION

The invention is based on the discovery of human GPCRs and their encoding cDNAs and on the use of the cDNA, or fragments thereof, and protein, or portions thereof, directly or as compositions for the diagnosis, prognosis, treatment and evaluation of therapies for neoplastic, neurological, and immune disorders, particularly follicular carcinoma of the thyroid, leiomyoma of the uterus, pancreatic cancer, epilepsy, interstitial nephritis, and immune response as a complication of cancer.

The cDNA encoding the human receptor of SEQ ID NO: 1 was first identified in Incyte Clone 1258981 from the brain meningioma cDNA library, through a computer-generated search for amino acid sequence alignments. The complete nucleotide sequence, SEQ ID NO:7, was assembled from the following overlapping and/or extended nucleic acid sequences: Incyte Clones 1258981H1 (MENITUT03), 1442823R1 (THYRNOT03), 1962119T6 (BRSTNOT04), 2059242R6 (OVARNOT03), and shotgun sequences, SATA01180F1, SARB01556F1, SARA01967F1, which are SEQ ID NOs:13-19, respectively.

The cDNA encoding the human GPCR of SEQ ID NO:2 was first identified in Incyte Clone 1459432 from the fetal colon cDNA library, through a computer-generated search for amino acid sequence alignments. The complete nucleotide sequence, SEQ ID NO:8, was assembled from the following overlapping and/or extended nucleic acid sequences: Incyte Clones 1459432H1 (COLNFET02), 1459432R1 (COLNFET02), 1459432×12 (COLNFET02), 3001554F6 (TLYMNOT06), and shotgun sequences, SAAC00257R1, SAAB00250R1, SAAB00523R1, which are SEQ ID NOs:20-26, respectively.

The cDNA encoding the human GPCR of SEQ ID NO:3 was first identified in Incyte Clone 2214673 from the fetal small intestine cDNA library, through a computer-generated search for amino PC-0044 CIP acid sequence alignments. The complete nucleotide sequence, SEQ ID NO:9, was assembled from the following overlapping and/or extended nucleic acid sequences: Incyte Clones 2214673H1 (SINTFET03), 3073644H1 (BONEUNT01), 3573501F6 (BRONNOT01), 4618526H1 (BRAYDIT01), 4857037H1 (BRSTTUT22), 5025086H1 (OVARNON03), and 1482004T1 (CORPNOT02) which are SEQ ID NOs:27-33, respectively.

The cDNA encoding the human GPCR of SEQ ID NO:4 was first identified in Incyte Clone 2488822 from the kidney tumor cDNA library, through a computer-generated search for amino acid sequence alignments. The complete nucleotide sequence, SEQ ID NO: 10, was assembled from the following overlapping and/or extended nucleic acid sequences: Incyte Clones 153210R6 (THP1PLB02), 2488822H1 (KIDNTUT13), 3558664T6 (LUNGNOT31), 2488822X308B1 (KIDNTUT13), and 2488822X310D1 (KIDNTUT13) which are SEQ ID NOs:34-38, respectively.

The cDNA encoding the human GPCR of SEQ ID NO:5 was first identified in Incyte Clone 2705201 from the cDNA library constructed from pons tissue affected by Alzheimer's disease through a computer-generated search for amino acid sequence alignments. The complete nucleotide sequence, SEQ ID NO: 11, was assembled from the following overlapping and/or extended nucleic acid sequences: Incyte Clones 2705201H1 (PONSAZT01), 3141184H1 (SMCCNOT02), 384797R6 (HYPONOB01), 2705201X325F1 (PONSAZT01), and 1262948X325F1 (SYNORAT05), which are SEQ ID NOs:39-43, respectively.

The cDNA encoding the human GPCR of SEQ ID NO:6 was first identified in Incyte Clone 3036563 from the PENCNOT02 cDNA library, through a computer-generated search for amino acid sequence alignments. The complete nucleotide sequence, SEQ ID NO: 12, was assembled from the following overlapping and/or extended nucleic acid sequences: Incyte Clones 3036563H1 (PENCNOT02), 4457161H1 (HEAADIR01), and shotgun sequences, SZAH00352F1, SZAH02656F1, SZAH01730F1, SZAH03622F1, SZAH01163F1, SZAH02669F1, SZAH00249F1, which are SEQ ID NOs:44-52, respectively.

Transcript imaging as shown in Example VIII details the specific and differential expression of SEQ ID NOs:7-12 in human disorders. In particular, the transcript images show that the nucleic acid sequence, protein or an antibody specific for the protein can be used in diagnostic assay for the following disorders:

| | |
|---|---|
| SEQ ID NO:7 | follicular carcinoma of the thyroid. |
| SEQ ID NO:8 | leiomyoma of the uterus. |
| SEQ ID NO:9 | cancerous pancreatic tissue |
| SEQ ID NO:10 | epilepsy |
| SEQ ID NO:11 | interstitial nephritis of the kidney |
| SEQ ID NO:12 | cytologically normal kidney |

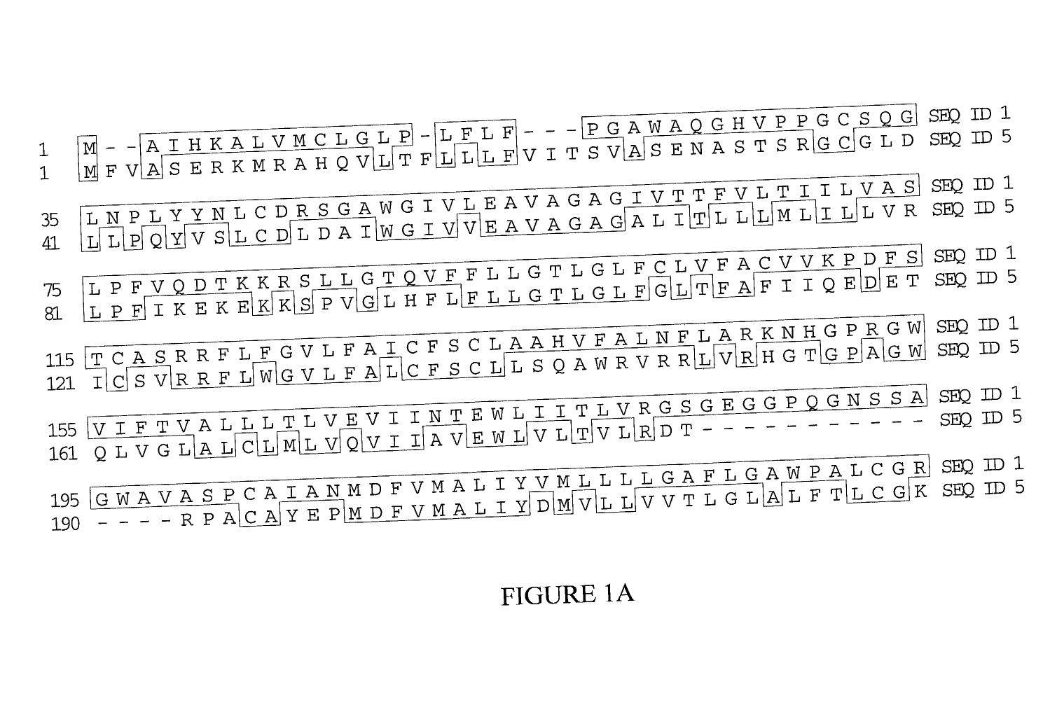

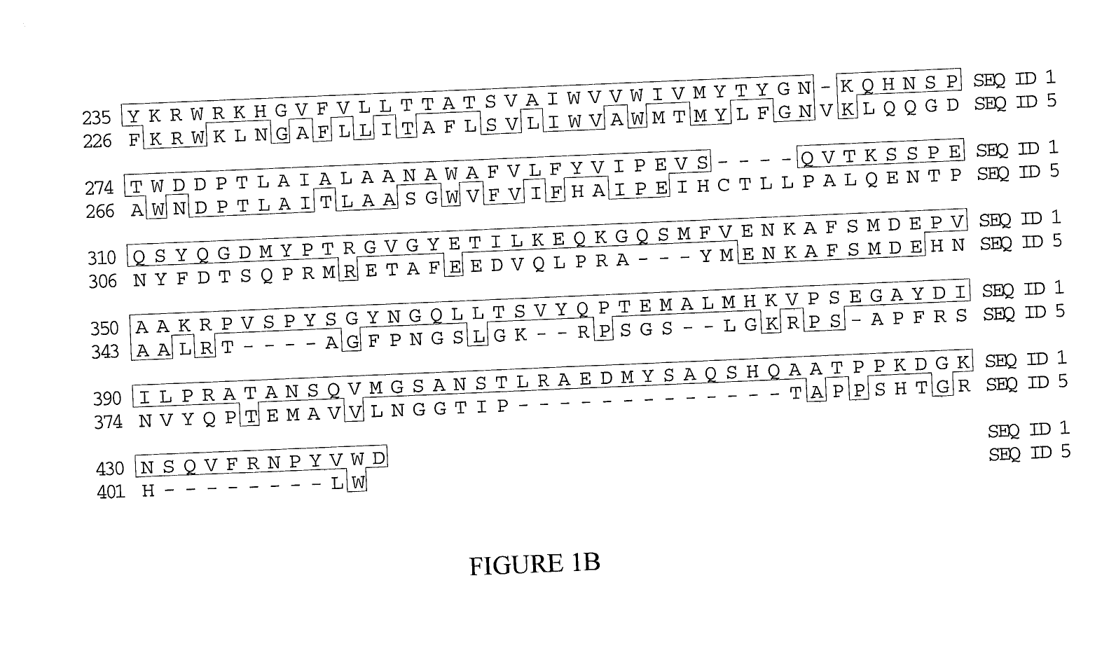

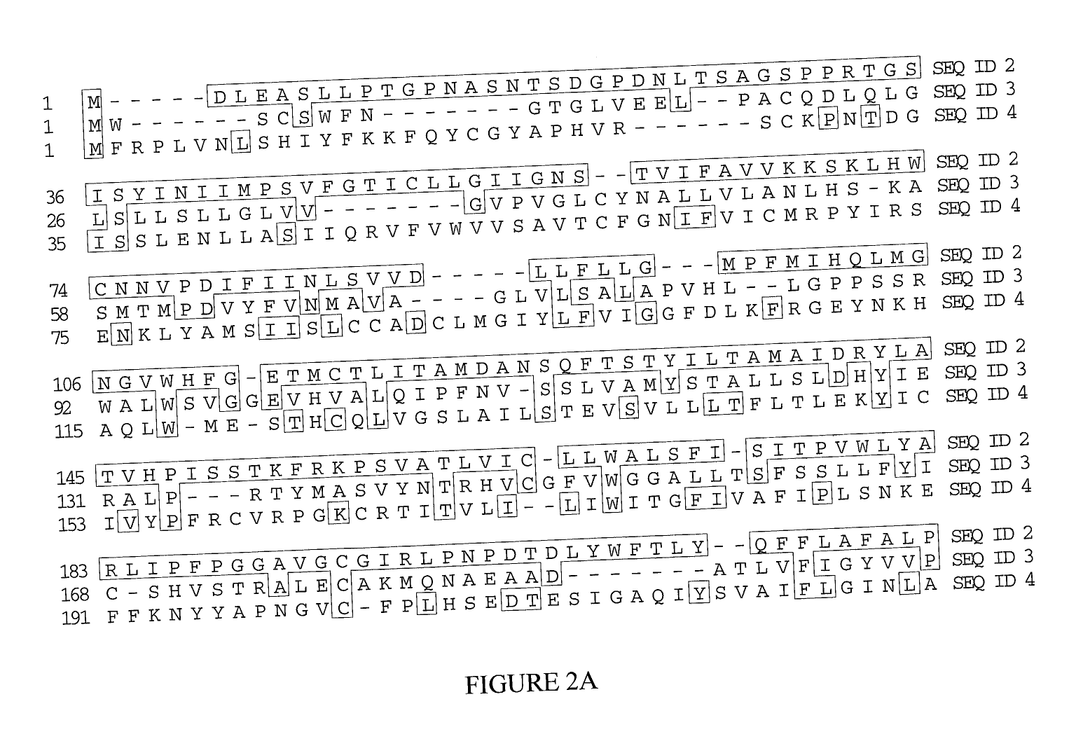

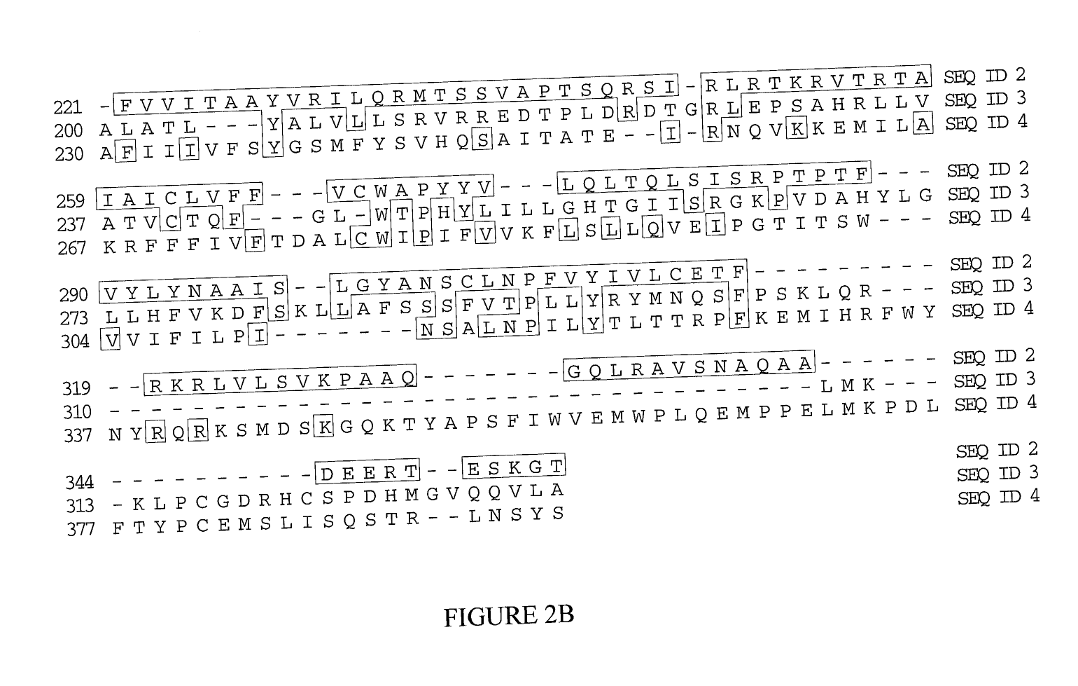

In one embodiment, the invention encompasses a polypeptide comprising a receptor having an amino acid sequence selected from SEQ ID NOs: 1-6 and characterized in Table 1 and shown in FIGS. 1 and 2. FIG. 1 displays the alignment of the metabotropic receptors, SEQ ID NO: 1 and 5, and FIG. 2, the alignment of the somatostatin and rhodopsin receptors. The signature sequences described in Table 2 are readily apparent in the alignments shown in FIGS. 1 and 2. For example, in FIG. 1, the transmembrane regions are clearly aligned in both receptors, SEQ ID NO: 1 at I51-V72 aligned with SEQ ID NO:5 at 157-L78; SEQ ID NO: 1 at G88-V109 aligned with SEQ ID NO:5 at G94-1115; SEQ ID NO: 1 at C116-A145 aligned with SEQ ID NO:5 at C122-V151; SEQ ID NO:1 at I156-L175 aligned with SEQ ID NO:5 at L162-L181; SEQ ID NO: 1 at M207-P229 aligned with SEQ ID NO:5 at M198-F220; and SEQ ID NO: 1 at G242-T264 aligned with SEQ ID NO:5 at G233-L255.

Mammalian variants of the cDNAs encoding the GPCRs were identified using BLAST2 with default parameters and the ZOOSEQ databases (Incyte Genomics, Palo Alto Calif.). These preferred variants have from about 84% to about 95% amino acid sequence identity to the human protein as shown in the table below. The first column shows the SEQ ID_{H} for the human cDNA; the second column, the SEQ ID_{VAR} for variant cDNAs; the third column, the clone numbers for the variants; the fourth column, the species; the fifth column, percent identity to the human cDNA; and the six column, the nucleotide alignment (Nt_{H}) of the human and variant cDNAs.

| SEQ ID_{H} | SEQ ID_{VAR} | Clone No. | Species | Identity | Nt_{H} Alignment |
|---|---|---|---|---|---|
| 7 | 53 | 702778992H2 | Dog | 91% | 805-1415 |
| 7 | 54 | 701938522F6 | Rat | 87% | 823-1378 |
| 8 | 55 | 700712581H1 | Monkey | 93% | 61-218 |
| 8 | 56 | 701250242H1 | Mouse | 90% | 386-656 |
| 8 | 57 | 701899983H1 | Rat | 89% | 625-928 |
| 8 | 58 | 701028051H1 | Rat | 84% | 170-417 |
| 9 | 59 | 075474_Mm.1 | Mouse | 88% | 478-878 |
| 9 | 60 | 700819903H1 | Rat | 85% | 559-736 |
| 9 | 61 | 701657796H1 | Rat | 84% | 787-1060 |
| 10 | 62 | 702466096T1 | Rat | 87% | 840-964 |
| 10 | 63 | 703021534H1 | Monkey | 95% | 12-703 |
| 10 | 64 | 703543565J1 | Dog | 87% | 1007-1450 |
| 11 | 65 | 076599_Mm.1 | Mouse | 85% | 14-243 |
| 11 | 66 | 701749639H1 | Rat | 89% | 321-874 |
| 11 | 67 | 702147192H1 | Rat | 86% | 23-515 |
| 12 | 68 | 703557532J1 | Dog | 88% | 2081-2491 |
| 12 | 69 | 702766139H1 | Dog | 81% | 125-509 |
| 12 | 70 | 701085654H2 | Mouse | 85% | 2083-2339 |
| 12 | 71 | 701077530H1 | Mouse | 86% | 1896-2096 |
| 12 | 72 | 702147631H1 | Rat | 86% | 1908-2264 |
| 12 | 73 | 702239655H1 | Rat | 85% | 1473-1995 |
| 12 | 74 | 702438348T1 | Rat | 87% | 2172-2398 |

It will be appreciated by those skilled in the art that as a result of the degeneracy of the genetic code, a multitude of cDNAs encoding each GPCR, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring GPCRs, and all such variations are to be considered as being specifically disclosed.

The cDNAs of SEQ ID NOs:7-74 may be used in hybridization, amplification, and screening technologies to identify and distinguish among SEQ ID NOs:7-12 and related molecules in a sample. The mammalian cDNAs, particularly SEQ ID NOs:53-74, may be used to produce transgenic cell lines or organisms which are model systems for human disorders including neoplastic, neurological and immune disorders upon which the toxicity and efficacy of potential therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

Characterization and Use of the Invention

cDNA Libraries

In a particular embodiment disclosed herein, mRNA is isolated from mammalian cells and tissues using methods which are well known to those skilled in the art and used to prepare the cDNA libraries. The Incyte cDNAs were isolated from mammalian cDNA libraries prepared as described in the EXAMPLES. The consensus sequences are chemically and/or electronically assembled from fragments including Incyte cDNAs and extension and/or shotgun sequences using computer programs such as PHRAP (P Green, University of Washington, Seattle Wash.), and the AUTOASSEMBLER application (Applied Biosystems, Foster City Calif.). After verification of the 5′ and 3′ sequence, at least one of the representative cDNAs which encode the receptor is designated a reagent. These reagent cDNAs are also used in the construction of human LIFEARRAYS (Incyte Genomics). A cDNA encoding at least a portion of each of the proteins of SEQ ID NOs: 1-4 and 6 are represented among the 17,096 sequences on HumanGenomeGEM1 (Incyte Genomics).

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech (APB), Piscataway N.J.), or combinations of polymerases and proofreading exonucleases such as those found in the ELONGASE amplification system (Life Technologies, Gaithersburg MD). Preferably, sequence preparation is automated with machines such as the MICROLAB 2200 system (Hamilton, Reno NV) and the DNA ENGINE thermal cycler (MJ Research, Watertown Mass.). Machines commonly used for sequencing include the ABI PRISM 3700, 377 or 373 DNA sequencing systems (Applied Biosystems (ABI), Foster City Calif.), the MEGABACE 1000 DNA sequencing system (APB), and the like. The sequences may be analyzed using a variety of algorithms well known in the art and described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, or deleted sequences can be removed or restored, respectively, organizing the incomplete assembled sequences into finished sequences.

Extension of a Nucleic Acid Sequence

The sequences of the invention may be extended using various PCR-based methods known in the art. For example, the XL-PCR kit (ABI), nested primers, and commercially available cDNA or genomic DNA libraries may be used to extend the nucleic acid sequence. For all PCR-based methods, primers may be designed using commercially available software, such as OLIGO primer analysis software (Molecular Biology Insights, Cascade Colo.) to be about 22 to 30 nucleotides in length, to have a GC content of about 50% or more, and to anneal to a target molecule at temperatures from about 55C to about 68C. When extending a sequence to recover regulatory elements, it is preferable to use genomic, rather than cDNA libraries.

Hybridization

The cDNA and fragments thereof can be used in hybridization technologies for various purposes.

A probe may be designed or derived from unique regions such as the 5′ regulatory region or from a 13 nonconserved region (i.e., 5′ or 3′ of the nucleotides encoding the conserved catalytic domain of the protein) and used in protocols to identify naturally occurring molecules encoding the receptors, allelic variants, or related molecules. The probe may be DNA or RNA, may be single-stranded, and should have at least 50% sequence identity to a nucleic acid sequence selected from SEQ ID NOs:7-74. Hybridization probes may be produced using oligolabeling, nick translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using commercially available kits.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60 C., which permits the formation of a hybridization complex between nucleic acid sequences that contain some mismatches. Subsequent washes are performed at higher stringency with buffers such as 0.2×SSC with 0.1% SDS at either 45 C. (medium stringency) or 68 C. (high stringency). At high stringency, hybridization complexes will remain stable only where the nucleic acids are completely complementary. In some membrane-based hybridizations, preferably 35% or most preferably 50%, formamide can be added to the hybridization solution to reduce the temperature at which hybridization is performed, and background signals can be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays incorporating cDNAs or antibodies may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Monoclonal or polyclonal antibodies may be used to detect or quantify expression of a protein in a sample. Such arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; Heller et al. (1997) U.S. PAT. NO. 5,605,662; and de Wildt et al. (2000) Nature Biotechnol 18:989-994.)

Hybridization probes are also useful in mapping the naturally occurring genomic sequence. The probes may be hybridized to a particular chromosome, a specific region of a chromosome, or an artificial chromosome construction. Such constructions include human artificial chromosomes (HAC), yeast artificial chromosomes (YAC), bacterial artificial chromosomes (BAC), bacterial P1 constructions, or the cDNAs of libraries made from single chromosomes. Expression Any one of a multitude of cDNAs encoding the receptors may be cloned into a vector and used to express the protein, or portions thereof, in host cells. The nucleic acid sequence can be engineered by such methods as DNA shuffling, as described in U.S. Pat. No. 5,830,721, and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mammalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional PBLUESCRIPT vector (Stratagene, La Jolla Calif.) or PSPORT1 plasmid (Life Technologies). Introduction of a nucleic acid sequence into the multiple cloning site of these vectors disrupts the lacZ gene and allows calorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6×His, FLAG, MYC, and the like. GST and 6-His are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-a-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the ABI 431A peptide synthesizer (ABI). A protein or portion thereof may be purified by preparative high performance liquid chromatography and its composition confirmed by amino acid analysis or by sequencing (Creighton (1984) Proteins, Structures and Molecular Properties, WH Freeman, New York N.Y.).

Preparation and Screening of Antibodies

Various hosts including, but not limited to, goats, rabbits, rats, mice, and human cell lines may be immunized by injection with an epitope selected using LASERGENE software and artificially synthesized or the receptors or any other immunogenic portion thereof as recombinantly produced . Adjuvants such as Freund's, mineral gels, and surface active substances such as lysolecithin, pluronic polyols, polyanions, peptides, oil emulsions, keyhole limpet hemacyanin (KLH), and dinitrophenol may be used to increase immunological response. The oligopeptide, peptide, or portion of protein used to induce antibodies should consist of at least about five amino acids, more preferably ten amino acids, which are identical to a portion of the natural protein. Oligopeptides may be fused with proteins such as KLH in order to produce antibodies to the chimeric molecule.

Monoclonal antibodies may be prepared using any technique which provides for the production of antibodies by continuous cell lines in culture. These include, but are not limited to, the hybridoma technique, the human B-cell hybridoma technique, and the EBV-hybridoma technique. (See, e.g., Kohler et al. (1975) Nature 256:495497; Kozbor et al. (1985) J. Immunol Methods 81:31-42; Cote et al. (1983) Proc Natl Acad Sci 80:2026-2030; and Cole et al. (1984) Mol Cell Biol 62:109-120.)

Alternatively, techniques described for antibody production may be adapted, using methods known in the art, to produce epitope-specific, single chain antibodies. Antibody fragments which contain specific binding sites for epitopes of the protein may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse et al. (1989) Science 246:1275-1281.)

The receptor, or a portion thereof, may be used in screening assays of phagemid or B-lymphocyte immunoglobulin libraries to identify antibodies having the desired specificity. Numerous protocols for competitive binding or immunoassays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering epitopes is preferred, but a competitive binding assay may also be employed (Pound (1998) Immunochemical Protocols, Humana Press, Totowa N.J.).

Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison Wis.) for incorporation of a labeled nucleotide such as ³²P-dCTP (APB), Cy3-dCTP or Cy5-dCTP (Operon Technologies, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

Diagnostics

Nucleic Acid Assays

The cDNAs, fragments, oligonucleotides, complementary RNA and DNA molecules, and PNAs may be used to detect and quantify differential gene expression for diagnostic purposes. Similarly antibodies which specifically bind a receptor of the invention may be used diagnostically, to quantitate protein expression. Disorders associated with specific and differential expression include neoplastic, neurological or immune disorders, particularly follicular carcinoma of the thyroid, leiomyoma of the uterus, pancreatic cancer, epilepsy, interstitial nephritis, and immune response as a complication of cancer. The diagnostic assay may use hybridization or amplification technology to compare gene expression in a biological sample from a patient to standard samples in order to detect differential gene expression. Qualitative or quantitative methods for this comparison are well known in the art.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Detection and quantification of a protein using either labeled amino acids or specific polyclonal or monoclonal antibodies are known in the art. Examples of such techniques include two-dimensional polyacrylamide gel electrophoresis, enzyme-linked immunosorbent assays (ELISAs), radioimmunoassays (RIAs), and fluorescence activated cell sorting (FACS). These assays and their quantitation against purifed, labeled standards are well known in the art (Ausubel, supra, unit 10.1-10.6). A two-site, monoclonal-based immunoassay utilizing monoclonal antibodies reactive to two non-interfering epitopes is preferred, but a competitive binding assay may be employed. (See, e.g., Coligan et al. (1997) Current Protocols in Immunology, Wiley-Interscience, New York N.Y.; and Pound, supra.)

Therapeutics

As described in THE INVENTION section, chemical and structural similarity in the sequence, signature sequences, specific motifs, or domains, exists among the receptors of FIG. 1 and FIG. 2. In addition, differential expression of these receptors is highly associated with neoplastic, neurological and immune disorders. The receptors clearly play a role in these disorders as shown in Example VIII.

In the treatment of cancer which is associated with the increased expression of the protein, it may be desirable to decrease protein expression or activity. In one embodiment, the an inhibitor, antagonist or antibody which specifically binds the protein may be administered to a subject to treat a condition associated with increased expression or activity. In another embodiment, a pharmaceutical composition comprising an inhibitor, antagonist, or antibody and a pharmaceutical carrier may be administered to a subject to treat a condition associated with the increased expression or activity of the endogenous protein. In an additional embodiment, a vector expressing the complement of the cDNA or fragments thereof may be administered to a subject to treat the disorder.

Any antisense molecules or vectors delivering these molecules may be administered in combination with other therapeutic agents. Selection of the agents for use in combination therapy may be made by one of ordinary skill in the art according to conventional pharmaceutical principles. A combination of therapeutic agents may act synergistically to affect treatment of a particular cancer at a lower dosage of each agent alone.

Modification of Gene Expression Using Nucleic Acids

Gene expression may be modified by designing complementary or antisense molecules (DNA, RNA, or PNA) to the control, 5′, 3′, or other regulatory regions of the gene encoding the receptors.

Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ -methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio-groups renders the molecule less available to endogenous endonucleases.

Screening and Purification Assays

The cDNAs encoding the receptors may be used to screen a library or a plurality of molecules or compounds for specific binding affinity. The libraries may be aptamers, DNA molecules, RNA molecules, PNAs, peptides, proteins such as transcription factors, enhancers, or repressors, and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the single-stranded or double-stranded molecule.

In one embodiment, the cDNA of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using a chaotropic agent to separate the protein from the purified ligand.

In a preferred embodiment, a GPCR may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify DNA molecules, RNA molecules, peptide nucleic acids, peptides, proteins, mimetics, agonists, antagonists, antibodies, immunoglobulins, inhibitors, and drugs or any other ligand, which specifically binds the protein.

In one aspect, this invention comtemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity, diagnostic, or therapeutic potential.

Pharmacology

Pharmaceutical compositions contain active ingredients in an effective amount to achieve a desired and intended purpose and a pharmaceutical carrier. The determination of an effective dose is well within the capability of those skilled in the art. For any compound, the therapeutically effective dose may be estimated initially either in cell culture assays or in animal models. The animal model is also used to achieve a desirable concentration range and route of administration. Such information may then be used to determine useful doses and routes for administration in humans.

A therapeutically effective dose refers to that amount of protein or inhibitor which ameliorates the symptoms or condition. Therapeutic efficacy and toxicity of such agents may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions which exhibit large therapeutic indexes are preferred. The data obtained from cell culture assays and animal studies are used in formulating a range of dosage for human use.

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gen, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a mammalian gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

I Tissue Descriptions and Construction of cDNA Libraries Tissues

The MENITUT03 library was constructed using RNA isolated from brain meningioma tissue removed from a 35-year-old Caucasian female during excision of a cerebral meningeal lesion. Pathology indicated a benign neoplasm in the right cerebellopontine angle of the brain. Patient history included hypothyroidism, and family history included myocardial infarction and breast cancer.

The COLNFET02 library was constructed using RNA isolated from the colon tissue of a Caucasian female fetus who died at 20 weeks gestation.

The SINTFET03 library was constructed using RNA isolated from kidney tumor tissue removed from a 5 1-year-old Caucasian female during a nephroureterectomy. Pathology indicated a grade 3 renal cell carcinoma. Patient history included depressive disorder, hypoglycemia, and uterine endometriosis, and family history included calculus of the kidney, colon cancer, and type II diabetes.

The PONSAZT01 library was constructed using RNA isolated from pons tissue removed from the brain of a 74-year-old Caucasian male who died from Alzheimer's disease.

The THP1PLB02 library was constructed by reamplification of THP1PLB01, which was made using RNA isolated from THP-1 cells cultured for 48 hours with 100 ng/ml phorbol ester (PMA), followed by a 4-hour culture in media containing 1 μg/ml LPS. THP-1 (ATCC TIB 202) is a human promonocyte line derived from the peripheral blood of a 1-year-old male with acute monocytic leukemia (ref: Int. J. Cancer (1980) 26:171).

The PENCNOT02 library was constructed using RNA isolated from right corpus cavernosum tissue of a penis.

Construction

RNA was isolated from the tissues described below. Some of the tissues were homogenized and lysed in guanidinium isothiocyanate; others were homogenized and lysed in phenol or a suitable mixture of denaturants, such as TRIZOL reagent (Life Technologies). The resulting lysates were centrifuged over CsCl cushions or extracted with chloroform. RNA was precipitated from the lysates with either isopropanol or sodium acetate and ethanol, or by other routine methods. Phenol extraction and precipitation of RNA were repeated as necessary to increase RNA purity.

In some cases, RNA was treated with DNAse. For most libraries, poly(A+) RNA was isolated using oligo d(T)-coupled paramagnetic particles (Promega, Madison Wis.), OLIGOTEX latex particles (Qiagen, Valencia Calif.), or an OLIGOTEX mRNA purification kit (Qiagen). Alternatively, RNA was isolated directly from lysates using RNA isolation kits such as the POLY(A)PURE mRNA purification kit; Ambion, Austin Tex.).

In some cases, Stratagene (La Jolla Calif.) was provided with RNA and constructed the cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6). Reverse transcription was initiated using oligo d(T) or random primers. Synthetic oligonucleotide adapters were ligated to double stranded cDNA, and the cDNA was digested with the appropriate restriction enzyme(s). For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of PBLUESCRIPT plasmid (Stratagene), pSPORT1 plasmid (Life Technologies), or pINCY plasmid (Incyte Genomics). Recombinant plasmids were transformed into competent E. coli cells including XL1-BLUE, XL1-BLUEMRF, or SOLR (Stratagene) or DH5α, DH10B, or ElectroMAX DH10B (Life Technologies).

II Isolation and Sequencing of CDNA Clones,

Plasmids were recovered from host cells by either in vivo excision using the UNIZAP vector system (Stratagene) or cell lysis. Plasmids were purified using one of the following kits or systems: a Magic or WIZARD Minipreps DNA purification system (Promega); a MINIPREP purification kit (Edge Biosystems, Gaithersburg Md.); and QIAWELL 8 plasmid, QIAWELL 8 Plus plasmid, QIAWELL 8 Ultra plasmid purification systems or the REAL PREP 96 plasmid kit (Qiagen). Following precipitation, plasmids were resuspended in 0.1 ml of distilled water and stored, with or without lyophilization, at 4C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene OR) and a Fluoroskan II fluorescence scanner (Labsystems Oy, Helsinki, Finland).

The cDNAs were prepared for sequencing using the CATALYST 800 preparation system (ABI) or the HYDRA microdispenser (Robbins Scientific) or MICROLAB 2200 system (Hamilton) systems in combination with the DNA ENGINE thermal cyclers (MJ Research). The cDNAs were sequenced using the ABI PRISM 373 or 377 sequencing systems (ABI) and standard ABI protocols, base calling software, and kits. In one alternative, cDNAs were sequenced using the MEGABACE 1000 DNA sequencing system (APB). In another alternative, the cDNAs were amplified and sequenced using the PRISM BIGDYE Terminator cycle sequencing ready reaction kit (ABI). In yet another alternative, cDNAs were sequenced using solutions and dyes from APB. Reading frames for the ESTs were determined using standard methods (reviewed in Ausubel, supra, unit 7.7).

The polynucleotide sequences derived from cDNA, extension, and shotgun sequencing were assembled and analyzed using a combination of software programs which utilize algorithms well known to those skilled in the art (Meyers, supra, pp 856-853) and described in Example IV.

III Extension of the Encoding Polynucleotides

The full length nucleic acid sequences of SEQ ID NO:7-12 were produced by extension of an appropriate fragment of the full length molecule using oligonucleotide primers designed from this fragment. One primer was synthesized to initiate 5′ extension of the known fragment, and the other primer, to initiate 3′ extension of the known fragment. The initial primers were designed using LASERGENE software (DNASTAR), or another appropriate program, to be about 22 to 30 nucleotides in length, to have a GC content of about 50% or more, and to anneal to the target sequence at temperatures of about 68C to about 72C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the DNA ENGINE thermal cyclers (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Life Technologies), and Pfu DNA polymerase (Stratagene), with the following parameters for primer pair PCI A and PCI B: Step 1: 94 C., 3 min; Step 2: 94 C., 15 sec; Step 3: 60 C., 1 min; Step 4: 68 C., 2 min; Step 5: Steps 2, 3, and 4 repeated 20 times; Step 6: 68 C., 5 min; Step 7: storage at 4 C. In the alternative, the parameters for primer pair T7 and SK+ were as follows: Step 1: 94 C., 3 min; Step 2: 94 C., 15 sec; Step 3: 57C., 1 min; Step 4: 68 C., 2 min; Step 5: Steps 2, 3, and 4 repeated 20 times; Step 6: 68 C., 5 min; Step 7: storage at 4 C.

The concentration of DNA in each well was determined by dispensing 100 μl PICOGREEN quantitation reagent (0.25% (v/v) PICOGREEN; Molecular Probes, Eugene Oreg.) dissolved in IX TE and 0.5 μl of undiluted PCR product into each well of an opaque fluorimeter plate (Corning Science Products, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (APB). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (APB), treated with Pfu DNA polymerase (Stratagene) to fill-in restriction site overhangs, and transfected into competent E. coli cells. Transformed cells were selected on antibiotic-containing media, individual colonies were picked and cultured overnight at 37 C. in 384-well plates in LB/2×carb liquid media.

The cells were lysed, and DNA was amplified by PCR using Taq DNA polymerase (APB) and Pfu DNA polymerase (Stratagene) with the following parameters: Step 1: 94 C., 3 min; Step 2: 94 C., 15 sec; Step 3: 60 C., 1 min; Step 4: 72 C., 2 min; Step 5: steps 2, 3, and 4 repeated 29 times; Step 6: 72 C., 5 min; Step 7: storage at 4 C. DNA was quantified by PICOGREEN reagent (Molecular Probes) as described above. Samples with low DNA recoveries were reamplified using the same conditions as described above. Samples were diluted with 20% dimethysulphoxide (1:2, v/v), and sequenced using DYENAMIC energy transfer sequencing primers and the DYENAMIC DIRECT kit (APB) or the ABI PRISM BIGDYE Terminator cycle sequencing ready reaction kit (ABI).

In like manner, the nucleotide sequences of SEQ ID NO:7-12 are used to obtain 5′ regulatory sequences using the procedure above, oligonucleotides designed for such extension, and an appropriate genomic library.

IV Homology Searching and Analysis of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST2 to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin and Altschul (1993; Proc Natl Acad Sci 90:5873-5877), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the stringency for an exact match was set from a lower limit of about 40 (with 1-2% error due to uncalled bases) to a 100% match of about 70.

The BLAST software suite (NCBI, Bethesda Md.; http://www.ncbi.nlm.nih.gov/gorf/bl2.html), includes various sequence analysis programs including “blastn” that is used to align nucleotide sequences and BLAST2 that is used for direct pairwise comparison of either nucleotide or amino acid sequences.

BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity is measured over the entire length of a sequence. Brenner et al. (1998; Proc Natl Acad Sci 95:6073-6078, incorporated herein by reference) analyzed BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another, and those having local similarity of at least 82% were combined and reassembled. Bins having templates with less than 95% local identity were split. Templates were subjected to analysis by STITCHER/EXON MAPPER algorithms that determine the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of <1×10-8. The templates were also subjected to frameshift FASTx against GENPEPT, and homolog match was defined as having an E-value of ≦1×10⁻⁸. Template analysis and assembly was described in U.S. SER. NO. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.; http://pfam.wustl.edu/). The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

V Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding a GPCR that have been mapped result in the assignment of all related regulatory and coding sequences to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VI Hybridization Technologies and Analyses

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton MA) by ultrasound in 0. 1% SDS and acetone, etching in 4% hydrofluoric acid (VWR Scientific Products, West Chester PA), coating with 0.05% aminopropyl silane (Sigma Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60 C.; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice, and used in membrane-based hybridizations as described below. Probe Preparation for Polymer Coated Slide Hybridization Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTT, 3 μl Cy3 or Cy5 labeling mix, 1 μl RNase inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85 C., and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/mil glycogen, 60 μl 5 M sodium acetate, and 300 μl 100% ethanol. The probe is centrifuged for 20 min at 20,800 ×g, and the pellet is resuspended in 12 μl resuspension buffer, heated to 65 C. for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester NY) is exposed to the membrane overnight at −70 C., developed, and examined visually.

Polymer Coated Slide-based Hybridization

Probe is heated to 65 C. for five min, centrifuged five min at 9400 rpm in a 5415 C. microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C. in 1×SSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer.

The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

VII Transcript Image

A transcript image was performed for SEQ ID NOs:7-12 at a product score of 70 using the LIFESEQ Gold database (rel Oct 00, Incyte Genomics). The transcript image allows assessment of the relative abundance of expressed cDNAs and their encoded proteins in one or more cDNA libraries. Criteria for transcript imaging include category, number of cDNAs per library, description of the library, and the like. All sequences and cDNA libraries in the database were categorized by system, organ/tissue, or cell type. The categories are Cardiovascular, Connective tissue Digestive, Embryonic structures, Endocrine, Exocrine glands, Female reproductive, Male reproductive, Germ cells, Hemic/immune system, Liver, Musculoskeletal, Nervous, Pancreas, Respiratory, Sense organs, Skin, Stomatognathic system, Unclassified/mixed, and Urinary tract.

For each category, the number of libraries in which the sequence was expressed were counted and shown over the total number of libraries in that category. In some transcript images, all normalized or pooled libraries, which have high copy number sequences removed prior to processing, and all mixed or pooled tissues, which are considered non-specific in that they contain more than one tissue type or more than one subject's tissue, can be excluded from the analysis. Cell lines and/or fetal tissue data can also be removed unless they serve as specific controls or represent possible consequences of inherited disorders and are the object of the investigation.

In the transcript images shown below, the first column lists the library name; the second column, the number of cDNAs sequenced for that library; the third column, the description of the tissue; the fourth column, abundance of the transcript; and the fifth column, percent abundance of the transcript.

SEQ ID NO:7
Category: Endocrine System

| Library* | cDNAs | Description | Abundance | % Abundance |
|---|---|---|---|---|
| THYRTUP02 | 457 | thyroid tumor, | 1 | 0.2188 |
| | | follicular CA, | | |
| | | CGAP | | |
| THYRNOT03 | 7173 | thyroid, | 4 | 0.0558 |
| | | mw/follicular | | |
| | | adenoma, 28F | | |
| THYRTMT01 | 3722 | thyroid, | 1 | 0.0269 |
| | | mw/papillary | | |
| | | CA, 56M | | |

*no libraries were excluded from this analysis

SEQ ID NO:7 was differentially expressed in follicular carcinoma of the thyroid. Expression was 4-fold higher than in any other thyroid tissue. In addition the sequence was not expressed in cytologically normal thyroid (5 libraries), lymphocytic thyroiditis (2 libraries), hyperthyroidism, goiter or papillary carcinoma. These data show that when used with biopsied thyroid tissue, SEQ ID NO:7 is diagnostic of thyroid tumor specifically follicular carcinoma.

SEQ ID NO:8
Category: Female Reproductive

| Library* | cDNAs | Description | Abundance | % Abundance |
|---|---|---|---|---|
| UTRSTUT07 | 2911 | uterus tumor, | 1 | 0.0344 |
| | | leiomyoma, | | |
| | | 41F | | |
| UTRSTUT04 | 3997 | uterus tumor, | 1 | 0.0250 |
| | | leiomyoma, | | |
| | | 34F | | |
| UTRSNOT02 | 13282 | uterus, | 1 | 0.0075 |
| | | aw/ovarian | | |
| | | follicular cysts, | | |
| | | 34F | | |

*16 normalized, pooled and mixed tissue libraries were excluded from this analysis.

SEQ ID NO:8 was differentially expressed in leiomyoma of the uterus. Expression was at least 3-fold higher than in any other uterine tissue. SEQ ID NO:8 distinguishes leiomyoma from adenosquamous carcinoma, endometrial adenocarcinoma, and serous papillary carcinoma and was not expressed in cervicitis (2 libraries), endometriosis (1 library), or cytologically normal endometrium (10 libraries), myometrium (6 libraries), or uterus (5 libraries).

SEQ ID NO:9
Category: Pancreas

| Library* | cDNAs | Description | Abundance | % Abundance |
|---|---|---|---|---|
| PANCNOT15 | 3638 | pancreas, islet | 1 | 0.0275 |
| | | cell | | |
| | | hyperplasia, | | |
| | | 15M | | |
| PANCNOT17 | 4034 | pancreas, mw/ | 1 | 0.0248 |
| | | neuroendocrine | | |
| | | CA, 65F | | |
| PANCTUP03 | 22651 | pancreas | 1 | 0.0044 |
| | | tumor, | | |
| | | adenoCA, | | |
| | | 3′ CGAP | | |

*no libraries were excluded from this analysis

SEQ ID NO:9 was specifically expressed in cancerous pancreatic tissue. SEQ ID NO:9 distiguishes islet cell hyperplasia, neuroendocrine carcinoma and pancreas tumor from diabetes and pancreatitis.

SEQ ID NO:10
Category: Nervous system

| Library* | cDNAs | Description | Abundance | % Abundance |
|---|---|---|---|---|
| BRAINOT03 | 5621 | brain, mw/ | 2 | 0.0356 |
| | | oligo- | | |
| | | astrocytoma, | | |
| | | epilepsy, 26M | | |
| BRAFNOT02 | 6394 | brain, frontal | 2 | 0.0313 |
| | | cortex, | | |
| | | aw/CHF, 35M | | |
| BRAINOT22 | 4980 | brain, | 1 | 0.0201 |
| | | temporal, | | |
| | | mw/tumor, | | |
| | | epilepsy, 45M | | |
| BRAINOT20 | 6302 | brain, | 1 | 0.0159 |
| | | temporal, | | |
| | | mw/epilepsy, | | |
| | | 27M | | |

SEQ ID NO: 10 was differentially expressed in brain in association with epilepsy. Among 221 libraries in the nervous system category, SEQ ID NO:10 was not expressed in Huntington's chorea, schizophrenia, Alzheimer's disease, multiple sclerosis, astrocytoma, meningioma, glioblastoma, other brain tumors or cytologically normal brain tissue.

SEQ ID NO:11
Category: Urinary Tract

| Library* | cDNAs | Description | Abundance | % Abundance |
|---|---|---|---|---|
| KIDPTDE01 | 3963 | kidney, | 3 | 0.0757 |
| | | interstitial | | |
| | | nephritis, | | |
| | | a63M, 5RP | | |
| KIDNTUP05 | 2690 | kidney tumor, | 1 | 0.0372 |
| | | renal cell, | | |
| | | 3′ CGAP | | |
| KIDNNOT25 | 3799 | kidney, | 1 | 0.0263 |
| | | mw/benign | | |
| | | cyst, | | |
| | | nephrolithiasis, | | |
| | | 42F | | |
| KIDNTUT14 | 3858 | kidney tumor, | 1 | 0.0259 |
| | | renal cell CA, | | |
| | | 43M, m/ | | |
| | | KIDNNOT20 | | |

*normalized, pooled, fetal and cell line libraries were excluded from this analysis

SEQ ID NO: 11 was differentially expressed in interstitial nephritis of the kidney. Expression was at least 2-fold higher than in any other kidney tissue. When used with biopsied kidney tissue, SEQ ID NO: 11 is diagnostic of interstitial nephritis which was clearly distinguishable from cytologically normal kidney tissues (12 libraries), renal cell carcinoma (7 libraries), benign cyst, and Wilms tumor (2 libraries).

SEQ ID NO:12
Category: Urinary Tract

| Library* | cDNAs | Description | Abundance | % Abundance |
|---|---|---|---|---|
| KIDNNOT26 | 3291 | kidney, | 3 | 0.0912 |
| | | medulla/cortex, | | |
| | | mw/renal cell | | |
| | | CA, 53F | | |
| KIDNNOT02 | 1977 | kidney, 64F | 1 | 0.0506 |
| KIDCTMT01 | 6142 | kidney, cortex, | 2 | 0.0326 |
| | | mw/renal cell | | |
| | | CA, 65M | | |
| KIDNNOT19 | 6952 | kidney, | 2 | 0.0288 |
| | | mw/renal cell | | |
| | | CA, 65M, m/ | | |
| | | KIDNTUT15 | | |
| KIDNNOT31 | 3507 | kidney | 1 | 0.0285 |
| KIDNNOT25 | 3799 | kidney, | 1 | 0.0263 |
| | | mw/benign | | |
| | | cyst, | | |
| | | nephrolithiasis, | | |
| | | 42F | | |
| KIDNNOT32 | 5619 | kidney, 49M | 1 | 0.0178 |

*pooled samples were not included in this analysis

SEQ ID NO: 12 is specifically expressed in cytologically normal kidney and is useful as a control in diagnostic tests for cancer, polycystic kidney disease, or other disorders of the kidney.

VIII Complementary Molecules

Molecules complementary to the cDNA, from about 5 (PNA) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

IX Expression of A Human GPCR

Expression and purification of the protein are achieved using either a mammalian cell expression system or an insect cell expression system. The pUB6/V5-His vector system (Invitrogen, Carlsbad Calif.) is used to express a GPCR in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6Δhis which enables purification as described above. Purified protein is used in the following activity and to make antibodies

X Production of Antibodies

A GPCR is purified using polyacrylamide gel electrophoresis and used to immunize mice or rabbits. Antibodies are produced using the protocols well known in the art and summarized below. Alternatively, the amino acid sequence of a GPCR is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 431A peptide synthesizer (ABI) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washng, and reacting with radio-iodinated goat anti-rabbit IgG. Methods well known in the art are used to determine antibody titer and the amount of complex formation.

XI Purification of Naturally Occurring Protein Using Specific Antibodies Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected. XII Screening Molecules for Specific Binding with the cDNA or Protein The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XIII Two-Hybrid Screen

A yeast two-hybrid system, MATCHMAKER LexA Two-Hybrid system (Clontech Laboratories, Palo Alto Calif.), is used to screen for peptides that bind the protein of the invention. A cDNA encoding the protein is inserted into the multiple cloning site of a pLexA vector, ligated, and transformed into E. coli. cDNA, prepared from MRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30 C. until the colonies have grown up and are counted. The colonies are pooled in a minimal volume of 1×TE (pH 7.5), replated on SDI-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

XIV Demonstration of Human GPCR Activity

GPCR activity of is determined in a ligand-binding assay using candidate ligand molecules in the presence of a protein selected from SEQ ID NOs: 1-6 and labeled with ¹²⁵I Bolton-Hunter reagent (Bolton et al. (1973) Biochem J 133:529-39) Candidate ligand molecules previously arrayed in the wells of a multiwell plate are incubated with the labeled protein, washed, and any wells with labeled protein:ligand complex are assayed. Data obtained using different concentrations of protein are used to calculate values for the number, affinity, and association of the protein with the ligand molecules.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| SEQ | Amino | Potential | Potential | | | |
|---|---|---|---|---|---|---|
| ID | Acid | Phosphorylation | Glycosylation | | | Analytical |
| NO: | Residues | Sites | Sites | Signature Sequences | Identification | Methods |
| 1 | 441 | S85 T164 T274 | N191 N405 | M1-A23, I51-V72, G88-P111 | Metabotropic | BLOCKS, HMM, |
| | | S306 S344 T81 | | C116-A145, I156-L175, | glutamate GPCR | MOTIFS, |
| | | S118 T407 Y312 | | M207-P229, G242-T264, | | PRINTS, SPSCAN |
| | | Y387 | | E330-K341 | | |
| 2 | 353 | S158 T255 S86 | N13 N16 N23 | I42-V66, P78-M99, | Somatostatin-like | BLAST, BLOCKS, |
| | | T120 S151 S243 | N58 N84 | W109-I149, V159-L180, | GPCR | HMM, MOTIFS, |
| | | S246 T251 T317 | | T209-L232, V254-T278, | | PFAM, PRINTS, |
| | | S325 | | Y293-R319 | | PROFILESCAN |
| 3 | 333 | T60 T218 S89 | N8 N110 N300 | Y44-L74, P62-H83, | Rhodopsin-like GPCR | BLAST, BLOCKS, |
| | | S172 T224 | | F109-R131, N143-L164, | | HMM, MOTIFS, |
| | | | | A231-G255, K278-P304 | | PFAM, PRINTS |
| 4 | 396 | S36 S187 T251 | N7 | I46-P70, Y79-I100, | Rhodopsin-like GPCR | BLAST, BLOCKS, |
| | | S27 T323 S389 | | L117-F157, R166-S187, | | HMM, MOTIFS, |
| | | | | S219-F242, L265-L289, | | PFAM, PRINTS, |
| | | | | S302-K328 | | PROFILESCAN |
| 5 | 403 | S360 S368 S47 | N30 N352 | I57-L78, G94-E117, | Metabotropic | BLOCKS, HMM, |
| | | T318 S337 S5 | | C122-V151, L162-L181, | glutamate GPCR | MOTIFS, PRINTS |
| | | T33 S123 T398 | | M198-F220, G233-L255 | | |
| 6 | 807 | T129 S155 S172 | N88 N110 N127 | N425-T452, I475-W499, | Secretin-like GPCR | BLAST, BLOCKS, |
| | | S201 S322 S347 | N281 N392 | A549-L572, F636-N647, | | HMM, MOTIFS, |
| | | S409 S662 S787 | N424 N443 | Q677-G696, H709-W730 | | PRINTS |
| | | S794 S117 T166 | N505 N647 | | | |
| | | T271 T402 T583 | N785 N798 | | | |
| | | T587 T618 S771 | | | | |

What is claimed is:

1. An isolated cDNA comprising a nucleic acid sequence encoding the amino acid sequence selected from SEQ ID NO: 1-6 or a complement of the encoding nucleic acid sequence.

2. An isolated cDNA comprising a nucleic acid sequence selected from:
a) SEQ ID NOs:7-12 and the complement thereof;
b) a fragment of SEQ ID NOs:7-12 selected from SEQ ID NOs:13-52 and the complements thereof; and
c) a variant of SEQ ID NOs:2 selected from SEQ ID NOs:53-74 and the complements thereof.

3. A composition comprising the cDNA of claim 1 and a labeling moiety.

4. A vector comprising the cDNA of claim 1.

5. A host cell comprising the vector of claim 4.

6. A method for using a cDNA to produce a protein, the method comprising:
a) culturing the host cell of claim 5 under conditions for protein expression; and
b) recovering the protein from the host cell culture.

7. A method for using a cDNA to detect differential expression of a nucleic acid in a sample comprising:
a) hybridizing the cDNA of claim 1 to the nucleic acids of the sample thereby forming at least one hybridization complex; and
b) detecting complex formation, wherein complex formation indicates differential expression in the sample.

8. The method of claim 7 further comprising amplifying the nucleic acids of the sample prior to hybridization.

9. The method of claim 7 wherein the cDNA is attached to a substrate.

10. The method of claim 7 wherein hybridization complexes are compared to at least one standard and are diagnostic of a squamous cell carcinoma.

11. A method of using a cDNA to screen a plurality of molecules or compounds, the method comprising:
a) combining the cDNA of claim 1 with a plurality of molecules or compounds under conditions to allow specific binding; and
b) detecting specific binding, thereby identifying a molecule or compound which specifically binds the cDNA.

12. The method of claim 11 wherein the molecules or compounds are selected from DNA molecules, RNA molecules, peptide nucleic acids, artificial chromosome constructions, peptides, transcription factors, repressors, and regulatory molecules.

13. A purified protein or a portion thereof comprising:
a) an amino acid sequence selected from SEQ ID NOs:1-6;
b) an antigenic epitope selected from SEQ ID NOs: 1-6; and
c) a biologically active portion of SEQ ID NOs: 1-6.

14. A composition comprising the protein of claim 13 and a labeling moiety or a pharmaceutical carrier.

15. A method for using a protein to screen a plurality of molecules or compounds to identify at least one ligand, the method comprising:
a) combining the protein of claim 13 with the molecules or compounds under conditions to allow specific binding; and
b) detecting specific binding, thereby identifying a ligand which specifically binds the protein.

16. The method of claim 15 wherein the molecules or compounds are selected from DNA molecules, RNA molecules, peptide nucleic acids, peptides, proteins, mimetics, agonists, antagonists, antibodies, immunoglobulins, inhibitors, and drugs.

17. A method of using a protein to prepare and purify antibodies comprising:
a) immunizing a animal with the protein of claim 13 under conditions to elicit an antibody response;
b) isolating animal antibodies;
c) attaching the protein to a substrate;
d) contacting the substrate with isolated antibodies under conditions to allow specific binding to the protein; c
e) dissociating the antibodies from the protein, thereby obtaining purified antibodies.

18. An antibody produced by the method of claim 17.

19. A method for using an antibody to detect expression of a protein in a sample, the method comprising:
a) combining the antibody of claim 18 with a sample under conditions which allow the formation of antibody:protein complexes; and
b) detecting complex formation, wherein complex formation indicates expression of the protein in the sample.

20. The method of claim 19 wherein expression is compared with standards and is diagnostic of cancer.


SEQUENCE LISTING (74 sequences)

SEQ ID NO: 1 - protein, 441 aa, Homo sapiens
  misc_feature: Incyte ID No 1258981CD1
MAIHKALVMCLGLPLFLFPGAWAQGHVPPGCSQGLNPLYYNLCDRSGAWGIVLEAVAGAGIVTTFVLTIILVASLPFVQDTKKRSLLGTQVFFLLGTLGLFCLVFACVVKPDFSTCASRRFLFGVLFAICFSCLAAHVFALNFLARKNHGPRGWVIFTVALLLTLVEVIINTEWLIITLVRGSGEGGPQGNSSAGWAVASPCAIANMDFVMALIYVMLLLLGAFLGAWPALCGRYKRWRKHGVFVLLTTATSVAIWVVWIVMYTYGNKQHNSPTWDDPTLAIALAANAWAFVLFYVIPEVSQVTKSSPEQSYQGDMYPTRGVGYETILKEQKGQSMFVENKAFSMDEPVAAKRPVSPYSGYNGQLLTSVYQPTEMALMHKVPSEGAYDIILPRATANSQVMGSANSTLRAEDMYSAQSHQAATPPKDGKNSQVFRNPYVWD

SEQ ID NO: 2 - protein, 353 aa, Homo sapiens
  misc_feature: Incyte ID No 1459432CD1
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPTSQRSIRLRTKRVTRTAIAICLVFFVCWAPYYVLQLTQLSISRPTPTFVYLYNAAISLGYANSCLNPFVYIVLCETFRKRLVLSVKPAAQGQLRAVSNAQAADEERTESKGT

SEQ ID NO: 3 - protein, 333 aa, Homo sapiens
  misc_feature: Incyte ID No 2214673CD1
MWSCSWFNGTGLVEELPACQDLQLGLSLLSLLGLVVGVPVGLCYNALLVLANLHSKASMTMPDVYFVNMAVAGLVLSALAPVHLLGPPSSRWALWSVGGEVHVALQIPFNVSSLVAMYSTALLSLDHYIERALPRTYMASVYNTRHVCGFVWGGALLTSFSSLLFYICSHVSTRALECAKMQNAEAADATLVFIGYVVPALATLYALVLLSRVRREDTPLDRDTGRLEPSAHRLLVATVCTQFGLWTPHYLILLGHTGIISRGKPVDAHYLGLLHFVKDFSKLLAFSSSFVTPLLYRYMNQSFPSKLQRLMKKLPCGDRHCSPDHMGVQQVLA

SEQ ID NO: 4 - protein, 396 aa, Homo sapiens
  misc_feature: Incyte ID No 2488822CD1
MFRPLVNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYAPNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

SEQ ID NO: 5 - protein, 403 aa, Homo sapiens
  misc_feature: Incyte ID No 2705201CD1
MFVASERKMRAHQVLTFLLLFVITSVASENASTSRGCGLDLLPQYVSLCDLDAIWGIVVEAVAGAGALITLLLMLILLVRLPFIKEKEKKSPVGLHFLFLLGTLGLFGLTFAFIIQEDETICSVRRFLWGVLFALCFSCLLSQAWRVRRLVRHGTGPAGWQLVGLALCLMLVQVIIAVEWLVLTVLRDTRPACAYEPMDFVMALIYDMVLLVVTLGLALFTLCGKFKRWKLNGAFLLITAFLSVLIWVAWMTMYLFGNVKLQQGDAWNDPTLAITLAASGWVFVIFHAIPEIHCTLLPALQENTPNYFDTSQPRMRETAFEEDVQLPRAYMENKAFSMDEHNAALRTAGFPNGSLGKRPSGSLGKRPSAPFRSNVYQPTEMAVVLNGGTIPTAPPSHTGRHLW

SEQ ID NO: 6 - protein, 807 aa, Homo sapiens
  misc_feature: Incyte ID No 3036563CD1
MGTYHCIFRYKNSYSIATKDVIVHPLPLKLNIMVDPLEATVSCSGSHHIKCCIEEDGDYKVTFHMGSSSLPAAKEVNKKQVCYKHNFNASSVSWCSKTVDVCCHFTNAANNSVWSPSMKLNLVPGENITCQDPVIGVGEPGKVIQKLCRFSNVPSSPESPIGGTITYKCVGSQWEEKRNDCISAPINSLLQMAKALIKSPSQDEMLPTYLKDLSISIGKAEHEISSSPGSLGAIINILDLLSTVPTQVNSEMMTHVLSTVNIILGKPVLNTWKVLQQQWTNQSSQLLHSVERFSQALQSGDSPPLSFSQTNVQMSSMVIKSSHPETYQQRFVFPYFDLWGNVVIDKSYLENLQSDSSIVTMAFPTLQAILAQDIQENNFAESLVMTTTVSHNTTMPFRISMTFKNNSPSGGETKCVFWNFRLANNTGGWDSSGCYVEEGDGDNVTCICDHLTSFSILMSPDSPDPSSLLGILLDIISYVGVGFSILSLAACLVVEAVVWKSVTKNRTSYMRHTCIVNIAASLLVANTWFIVVAAIQDNRYILCKTACVAATFFIHFFYLSVFFWMLTLGLMLFYRLVFILHETSRSTQKAIAFCLGYGCPLAISVITLGATQPREVYTRKNVCWLNWEDTKALLAFAIPALIIVVVNITITIVVITKILRPSIGDKPCKQEKSSLFQISKSIGVLTPLLGLTWGFGLTTVFPGTNLVFHIIFAILNVFQGLFILLFGCLWDLKVQEALLNKFSLSRWSSQHSKSTSLGSSTPVFSMSSPISRRFNNLFGKTGTYNVSTPEATSSSLENSSSASSLLN

SEQ ID NO: 7 - DNA, 1819 bp, Homo sapiens
  misc_feature: Incyte ID No 1258981CB1
cggctcgagccctcaccagccggaaagtacgagtcggctcagcctggagggacccaaccagagcctggcctgggagccaggatggccatccacaaagccttggtgatgtgcctgggactgcctctcttcctgttcccaggggcctgggcccagggccatgtcccacccggctgcagccaaggcctcaaccccctgtactacaacctgtgtgaccgctctggggcgtggggcatcgtcctggaggccgtggctggggcgggcattgtcaccacgtttgtgctcaccatcatcctggtggccagcctcccctttgtgcaggacaccaagaaacggagcctgctggggacccaggtattcttccttctggggaccctgggcctcttctgcctcgtgtttgcctgtgtggtgaagcccgacttctccacctgtgcctctcggcgcttcctctttggggttctgttcgccatctgcttctcttgtctggcggctcacgtctttgccctcaacttcctggcccggaagaaccacgggccccggggctgggtgatcttcactgtggctctgctgctgaccctggtagaggtcatcatcaatacagagtggctgatcatcaccctggttcggggcagtggcgagggcggccctcagggcaacagcagcgcaggctgggccgtggcctccccctgtgccatcgccaacatggactttgtcatggcactcatctacgtcatgctgctgctgctgggtgccttcctgggggcctggcccgccctgtgtggccgctacaagcgctggcgtaagcatggggtctttgtgctcctcaccacagccacctccgttgccatatgggtggtgtggatcgtcatgtatacttacggcaacaagcagcacaacagtcccacctgggatgaccccacgctggccatcgccctcgccgccaatgcctgggccttcgtcctcttctacgtcatccccgaggtctcccaggtgaccaagtccagcccagagcaaagctaccagggggacatgtaccccacccggggcgtgggctatgagaccatcctgaaagagcagaagggtcagagcatgttcgtggagaacaaggccttttccatggatgagccggttgcagctaagaggccggtgtcaccatacagcgggtacaatgggcagctgctgaccagtgtgtaccagcccactgagatggccctgatgcacaaagttccgtccgaaggagcttacgacatcatcctcccacgggccaccgccaacagccaggtgatgggcagtgccaactcgaccctgcgggctgaagacatgtactcggcccagagccaccaggcggccacaccgccgaaagacggcaagaactctcaggtctttagaaacccctacgtgtgggactgagtcagcggtggcgaggagaggcggtcggatttggggagggccctgaggacctggccccgggcaagggactctccaggctcctcctccccctggcaggcccagcaacatgtgccccagatgtggaagggcctccctctctgccagtgtttgggtgggtgtcatgggtgtccccacccactcctcagtgtttgtggagtcgaggagccaaccccagcctcctgccaggatcacctcggcggtcacactccagccaaatagtgttctcggggtggtggctgggcagcgcctatgtttctctggagattcctgcaacctcaagagacttcccaggcgctcaggcctggatcttgctcctctgtgaggaacaagggtgcctaataaatacatttctgctttattaactcttaaaaaa

SEQ ID NO: 8 - DNA, 2138 bp, Homo sapiens
  misc_feature: Incyte ID No 1459432CB1
ttatgtctggtcgactctgaattgggcttggaggcggcacggctgccaggctacggaggtagacccccttcccaactgcggggcttgcgctccgggacaaggtggcaggcgctggaggctgccgcagcctgcgtgggtggaggggagctcagctcggttgtggcagcatgcgaccggcactggctggatggacctggaagcctcgctgctgcccactggtcccaatgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccacctcccagcgcagcatccggctgcggacaaagagggtgacccgcacagccatcgccatctgtctggtcttctttgtgtgctgggcaccctactatgtgctacagctgacccagttgtccatcagccgcccgacccccacctttgtctacttatacaatgcggccatcagcttgggctatgccaacagctgcctcaacccgtttgtgtacatcgtgctctgtgagacgttccgcaaacgcttggtcctgtcggtgaagcctgcagcccaggggcagcttcgcgctgtcagcaacgctcaggcggctgacgaggagaggacagaaagcaaaggcacctgatacttcccctgccaccctgcacacctccaagtcagggcaccacaacacgccaccgggagagatgctgagaaaaacccaagaccgctcgggaaatgcaggaaggccgggttgtgaggggttgttgcaatgaaataaatacattccatggggctcacacgttgctggggaggcctggagtcaggtttggggttttcagatatcagaaatccccttgggggagcaggatgagacctttggatagaacagaagctgagcaagagaacatgttggtttggataaccggttgcactatatctgtgagctctcaaatgtcttcttcccaaggcaagaggtggaagggtactgactgggtttgtttaaagtcaggcagggctggagtgagcagccagggccatgttgcacaaggcctgagagacgggaaagggcccgatcgctctttcccgcctctcactggtgcgatggaaggtggcctttctcccaagctggtggataatgaaaaataaagcatcccatctctcggcgttccagcatcctgtcaatttcccttttgctctagaggatgcatgtttatttgaggggatgtggcactgagcccacaggagtaaaagcccagtttgctaggaggtctgcttactgaaaacaaggagacctggggtgggtgtggttgggggtcttaaaactaataaaagctggggtcggggggcttttgcagctctggtgacattctctccacggggcacatttgctcagtcactaatccagcttgagtgtccgtgtgttctgcatgtgcaggggtcattctagtgcccggtgtgttggcatcatctttttgctctagcccttcctctccaaaataaaatcaaataaaggaaaatctccacccaaaaaa

SEQ ID NO: 9 - DNA, 1878 bp, Homo sapiens
  misc_feature: Incyte ID No 2214673CB1
cgcacagcgcgcaggtcctcaccagagctctggtggccacctctgtcccgccatgctgctcaccgacagtggccagggcccacagcaccaagaggcttgggccacaaagtaaagggtcgcggagcctcgccggccgccatgtggagctgcagctggttcaacggcacagggctggtggaggagctgcctgcctgccaggacctgcagctggggctgtcactgttgtcgctgctgggcctggtggtgggcgtgccagtgggcctgtgctacaacgccctgctggtgctggccaacctacacagcaaggccagcatgaccatgccggacgtgtactttgtcaacatggcagtggcaggcctggtgctcagcgccctggcccctgtgcacctgctcggccccccgagctcccggtgggcgctgtggagtgtgggcggcgaagtccacgtggcactgcagatccccttcaatgtgtcctcactggtggccatgtactccaccgccctgctgagcctcgaccactacatcgagcgtgcactgccgcggacctacatggccagcgtgtacaacacgcggcacgtgtgcggcttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgcacgcagtttgggctctggacgccacactatctgatcctgctggggcacacgggcatcatctcgcgagggaagcccgtggacgcacactacctggggctactgcactttgtgaaggatttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgtaggcggcccagccctcctggggagacgtgactctggtggacgcagagcacttagttaccctggacgctccccacatccttccagaaggagacgagctgctggaagagaagcaggaggggtgtttttcttgaagtttcctttttcccacaaatgccactcttgggccaaggctgtggtccccgtggctggcatctggcttgagtctccccgaggcctgtgcgtctcccaaacacgcagctcaaggtccacatccgcaaaagcctcctcgccttcagcctcctcagcattcagtttgtcaatgaagtgatgaaagcttagagccagtatttatactttgtggttaaaatacttgattcccccttgtttgttttacaaaaacagatgtttcctagaaaaatgacaaatagtaaaatgaacaaaaccctacgaaagaatggcaacagccagggtggccgggccctgccagtgggcggcgtgtgctagcaaggcctgccgggtgtgccgcagtcaccacagggttctgagaacatttcacagaagtgcctgagacgcggagacatggctggtgttaaatggagctattcaatagcagtgacgcgctctcctcagccaccaaatgtccctgacaccctccccagcccccacagataacatcagctgaggtttttttcagtatgaacctgtcctaaatcaattcctcaaagtgtgcacaaaactaaagaatataaataaacagaagc

SEQ ID NO: 10 - DNA, 1804 bp, Homo sapiens
  misc_feature: Incyte ID No 2488822CB1
taagtgttaactaaaagcattttattaaattgtccttcacagaaactcaatttattaaaccatgtataatacatgttcctttgattgattattaatttgatatttttagcagcctagaagggattgaaatttcaaatatccaacaaaggatgtttagacctcttgtgaatctctctcacatatattttaagaaattccagtactgtgggtatgcaccacatgttcgcagctgtaaaccaaacactgatggaatttcatctctagagaatctcttggcaagcattattcagagagtatttgtctgggttgtatctgcagttacctgctttggaaacatttttgtcatttgcatgcgaccttatatcaggtctgagaacaagctgtatgccatgtcaatcatttctctctgctgtgccgactgcttaatgggaatatatttattcgtgatcggaggctttgacctaaagtttcgtggagaatacaataagcatgcgcagctgtggatggagagtactcattgtcagcttgtaggatctttggccattctgtccacagaagtatcagttttactgttaacatttctgacattggaaaaatacatctgcattgtctatccttttagatgtgtgagacctggaaaatgcagaacaattacagttctgattctcatttggattactggttttatagtggctttcattccattgagcaataaggaatttttcaaaaactactatgcacccaatggagtatgcttccctcttcattcagaagatacagaaagtattggagcccagatttattcagtggcaatttttcttggtattaatttggccgcatttatcatcatagttttttcctatggaagcatgttttatagtgttcatcaaagtgccataacagcaactgaaatacggaatcaagttaaaaaagagatgatccttgccaaacgttttttctttatagtatttactgatgcattatgctggatacccatttttgtagtgaaatttctttcactgcttcaggtagaaataccaggtaccataacctcttgggtagtgatttttattctgcccattaacagtgctttgaacccaattctctatactctgaccacaagaccatttaaagaaatgattcatcggttttggtataactacagacaaagaaaatctatggacagcaaaggtcagaaaacatatgctccatcattcatctgggtggaaatgtggccactgcaggagatgccacctgagttaatgaagccggaccttttcacatacccctgtgaaatgtcactgatttctcaatcaacgagactcaattcctattcatgactgactctgaaattcatttcttcgcagagaatactgtgggggtgcttcatgagggatttactggtatgaaatgaataccacaaaattaatttataataatagctaagataaatattttacaaggacatgaggaaaaataaaaatgactaatgctcttacaaagggaagtaattatatcaataatgtatatatattagtagacattttgcataagaaattaagagaaatctacttcagtaacattcattcatttttctaacatgcatttattgagtacccactactatgtgcatagcattgcaatatagtcctggaagtagacagtgcagaacctttcaatctgtagatggtgtttaatgacaaaagactatacaaagtccatctgcagttcctagtttaaagtagagctttacctgtcatgtgcatcagcaagaatcatagcgattttaaatagaggtgtggac

SEQ ID NO: 11 - DNA, 1515 bp, Homo sapiens
  misc_feature: Incyte ID No 2705201CB1
tgccgaagagtctggagcgtcggcgctgcggggccgcgggggtcgaatgttcgtggcatcagagagaaagatgagagctcaccaggtgctcaccttcctcctgctcttcgtgatcacctcggtggcctctgaaaacgccagcacatcccgaggctgtgggctggacctcctccctcagtacgtgtccctgtgcgacctggacgccatctggggcattgtggtggaggcggtggccggggcgggcgccctgatcacactgctcctgatgctcatcctcctggtgcggctgcccttcatcaaggagaaggagaagaagagccctgtgggcctccactttctgttcctcctggggaccctgggcctctttgggctgacgtttgccttcatcatccaggaggacgagaccatctgctctgtccgccgcttcctctggggcgtcctctttgcgctctgcttctcctgcctgctgagccaggcatggcgcgtgcggaggctggtgcggcatggcacgggccccgcgggctggcagctggtgggcctggcgctgtgcctgatgctggtgcaagtcatcatcgctgtggagtggctggtgctcaccgtgctgcgtgacacaaggccagcctgcgcctacgagcccatggactttgtgatggccctcatctacgacatggtactgcttgtggtcaccctggggctggccctcttcactctgtgcggcaagttcaagaggtggaagctgaacggggccttcctcctcatcacagccttcctctctgtgctcatctgggtggcctggatgaccatgtacctcttcggcaatgtcaagctgcagcagggggatgcctggaacgaccccaccttggccatcacgctggcggccagcggctgggtcttcgtcatcttccacgccatccctgagatccactgcacccttctgccagccctgcaggagaacacgcccaactacttcgacacgtcgcagcccaggatgcgggagacggccttcgaggaggacgtgcagctgccgcgggcctatatggagaacaaggccttctccatggatgaacacaatgcagctctccgaacagcaggatttcccaacggcagcttgggaaaaagacccagtggcagcttggggaaaagacccagcgctccgtttagaagcaacgtgtatcagccaactgagatggccgtcgtgctcaacggtgggaccatcccaactgctccgccaagtcacacaggaagacacctttggtgaaagactttaagttccagagaatcagaatttctcttaccgatttgcctccctggctgtgtctttcttgagggagaaatcggtaacagttgccgaaccaggccgcctcacagccaggaaatttggaaatcctagccaaggggatttcgtgtaaatgtgaacactgacgaactgaaaagctaacaccgactgcccgcccctcccctgccacacacacagacacgtaataccagaccaacctcaatccccaccttaaaaaa

SEQ ID NO: 12 - DNA, 2919 bp, Homo sapiens
  misc_feature: Incyte ID No 3036563CB1
atcttgatggagcagaatcagtactgacagtcaagacctcgaccagggagtggaatgggaacctatcactgcatatttagatataagaattcatacagtattgcaaccaaagacgtcattgttcacccgctgcctctaaagctgaacatcatggttgatcctttggaagctactgtttcatgcagtggttcccatcacatcaagtgctgcatagaggaggatggagactacaaagttactttccatatgggttcctcatcccttcctgctgcaaaagaagttaacaaaaaacaagtgtgctacaaacacaatttcaatgcaagctcagtttcctggtgttcaaaaactgttgatgtgtgttgtcactttaccaatgctgctaataattcagtctggagcccatctatgaagctgaatctggttcctggggaaaacatcacatgccaggatcccgtaataggtgtcggagagccggggaaagtcatccagaagctatgccggttctcaaacgttcccagcagccctgagagtcccattggcgggaccatcacttacaaatgtgtaggctcccagtgggaggagaagagaaatgactgcatctctgccccaataaacagtctgctccagatggctaaggctttgatcaagagcccctctcaggatgagatgctccctacatacctgaaggatctttctattagcataggcaaagcggaacatgaaatcagctcttctcctgggagtctgggagccattattaacatccttgatctgctctcaacagttccaacccaagtaaattcagaaatgatgacgcacgtgctctctacggttaatatcatccttggcaagcccgtcttgaacacctggaaggttttacaacagcaatggaccaatcagagttcacagctactacattcagtggaaagattttcccaagcattacagtcaggagatagccctccattgtccttctcccaaactaatgtgcagatgagcagcatggtaatcaagtccagccacccagaaacctatcaacagaggtttgttttcccatactttgacctctggggcaatgtggtcattgacaagagctacctagaaaacttgcagtcggattcgtctattgtcaccatggctttcccaactctccaagccatccttgctcaggatatccaggaaaataactttgcagagagcttagtgatgacaaccactgtcagccacaatacgactatgccattcaggatttcaatgacttttaagaacaatagcccttcaggcggcgaaacgaagtgtgtcttctggaacttcaggcttgccaacaacacaggggggtgggacagcagtgggtgctatgttgaagaaggtgatggggacaatgtcacctgtatctgtgaccacctaacatcattctccatcctcatgtcccctgactccccagatcctagttctctcctgggaatactcctggatattatttcttatgttggggtgggcttttccatcttgagcttggcagcctgtctagttgtggaagctgtggtgtggaaatcggtgaccaagaatcggacttcttatatgcgccacacctgcatagtgaatatcgctgcctcccttctggtcgccaacacctggttcattgtggtcgctgccatccaggacaatcgctacatactctgcaagacagcctgtgtggctgccaccttcttcatccacttcttctacctcagcgtcttcttctggatgctgacactgggcctcatgctgttctatcgcctggttttcattctgcatgaaacaagcaggtccactcagaaagccattgccttctgtcttggctatggctgcccacttgccatctcggtcatcacgctgggagccacccagccccgggaagtctatacgaggaagaatgtctgttggctcaactgggaggacaccaaggccctgctggctttcgccatcccagcactgatcattgtggtggtgaacataaccatcactattgtggtcatcaccaagatcctgaggccttccattggagacaagccatgcaagcaggagaagagcagcctgtttcagatcagcaagagcattggggtcctcacaccactcttgggcctcacttggggttttggtctcaccactgtgttcccagggaccaaccttgtgttccatatcatatttgccatcctcaatgtcttccagggattattcattttactctttggatgcctctgggatctgaaggtacaggaagctttgctgaataagttttcattgtcgagatggtcttcacagcactcaaagtcaacatccctgggttcatccacacctgtgttttctatgagttctccaatatcaaggagatttaacaatttgtttggtaaaacaggaacgtataatgtttccaccccagaagcaaccagctcatccctggaaaactcatccagtgcttcttcgttgctcaactaagaacaggataatccaacctacgtgacctcccggggacagtggctgtgcttttaaaaagagatgcttgcaaagcaatggggaacgtgttctcggggcaggtttccgggagcagatgccaaaaagactttttcatagagaagaggctttcttttgtaaagacagaataaaaataattgttatgtttctgtttgttccctccccctcccccttgtgtgataccacatgtgtatagtatttaagtgaaactcaagccctcaaggcccaacttctctgtctatattgtaatatagaatttcgaagagacattttcactttttacacattgggcacaaagataagctttgattaaagtagtaagtaaaaggctacctaggaaatacttcagtgaattctaagaaggaaggaaggaaggaaggagggaaagaagggaggaaaccagga

SEQ ID NO: 13 - DNA, 232 bp, Homo sapiens
  misc_feature: Incyte ID No 1258981H1
tgtcaccatacagcgggtacaatgggcagctgctgaccagtgtgtaccagcccactgagatggccctgatgcacaaagntccgtccnaangagcttacgacatcatcctcccacgggccatcgccaacagccaggtgatgggcagtgcnaactcgaccctgngggctgaagacatgtactcggcccagngccaccaggnggncanaccgccgaaaganggcaagaactctct

SEQ ID NO: 14 - DNA, 516 bp, Homo sapiens
  misc_feature: Incyte ID No 1442823R1
aagagttaataaagcagaaatgtatttattaggcacccttgttcctcacagaggagcaagatccaggcctgagcgcctgggaagtctcttgaggttgcaggaatctccagagaaacataggcgctgcccagccaccaccccgagaacactatttggctggagtgtgaccgccgaggtgatcctggcaggaggctggggttggctcctcgactccacaaacactgaggagtgggtggggacacccatgacacccacccaaacactggcagagagggaggcccttccacatctggggcacatgttgctgggcctgccagggggaggaggagcctggagagtcccttgcccggggccaggtcctcagggccctccccaaatccgaccgcctctcctcgccaccgctgactcagtcccacacgtaggggtttctaaagacctgagagttcttgccgtctttcggcggtgtggcgcctggtggctctgggccgagtacatgtcttcagcccgcaggtcgag

SEQ ID NO: 15 - DNA, 268 bp, Homo sapiens
  misc_feature: Incyte ID No 1962119T6
cacagaggagcaagatccaggcctgagcgcctgggaagtctcttgaggttgcaggaatctccagagaaacataggcgctgcccagccaccaccccgagaacactatttggctggagtgtgaccgccgaggtgatcctggcaggaggctggggttggctcctcgactccacaaacactgaggagtgggtggggacacccatgacacccacccaaacactggcagagagggaggcccttccacatctggggcacatgttgctgggcctgc

SEQ ID NO: 16 - DNA, 246 bp, Homo sapiens
  misc_feature: Incyte ID No 2059242R6
cagtgtttgggtgggtgtcatgggtgtccccacccactcctcagtgtttgtggagtcgaggagccaaccccagcctcctgccaggatcacctcggcggtcacactccagccaaatagtgttctcggggtggtggctgggcagcgcctatgtttctctggagattcctgcaacctcaagagacttcccaggcgctcaggcctggatcttgctcctctgtgaggaacaagggtgcctaataaatacat

SEQ ID NO: 17 - DNA, 300 bp, Homo sapiens
  misc_feature: Incyte ID No SATA01180F1
gactctagaggatcccccttcaccacacaggcaaacacgaggcagaagangnccanggtccccagnaagaagaatacctgggtccccagcaggctccgtttcttggtgtcctgcacaaaggggaggctggccaccaggatgatggtgagcacaaacgtggtgacaatgcccgccccagccacggcctccaggacgatgccccacgccccagagcggtcacacaggttgtagtncagggggttgaggccttggctgcagccgggtgggacatnggggtaccgagctcgaattcgtantcat

SEQ ID NO: 18 - DNA, 467 bp, Homo sapiens
  misc_feature: Incyte ID No SARB01556F1
cctgcaggtcgactctagaggataggcctcacgtctttgcnctcaacttcntggcccggaagaaccacgggccccggggctgggtgannttcactgtggctctgntgctgaccctggtagaggtcannatcaatacagagtggctgatcatcaccctggttcggggcagtggnganggcggccctcagggcaacagcagnncaggctnggccgtggnntcncnctgtgnnatcgnnaancatggatttgtnatagcactnatctcacgtnatgntgntgntgctgggtgccttcntgggggcctggnncagcnnctgtgttggcngctaaagccctggngtaagaatggggtctttgtngtnntcaanaaaaccanctcngntgccatatnggtagtgagaaacnncangtatnnntacaggcaacaagcacccnnaacantttccannctgggnangnacccaaag

SEQ ID NO: 19 - DNA, 631 bp, Homo sapiens
  misc_feature: Incyte ID No SARA01967F1
atccatggaaaaggccttgttctccacgaacatgctctgacccttctgctctttcaggatggtctcatagcccacgccccgggtggggtacatgtccccctggtagctttgctctgggctggacttggtcacctgggagacctcggggatgacgtagaagaggacgaaggcccaggcattggcggcgagggcgatggccagcgtggggtcatcccaggtgggactgttntgctgcttgtngccgtaagtatacatgacgatccacaccacccatatggcaacggaggtggctgtggtgaggagcacaaagaccccatgcttacgccagcgcttgtagcggncacacagggcgggccaggcccccaggaaggcacccagcagcagcagcatgacgtagatgagtgccaatgncaaagtccatgttggcgatggcacaagggggganggccaagggccccagggggnnacngaggcttngaaatttggtaaanncaaggtnnaaaancaagntttcccnngggngnnaaaaattttttaanncccgncnncanaaatttccccanncangananntttanngatccngggaaancccataaaaaaantnttaaaaaccccttggggggnnccc

SEQ ID NO: 20 - DNA, 223 bp, Homo sapiens
  misc_feature: Incyte ID No 1459432H1
ggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccag

SEQ ID NO: 21 - DNA, 475 bp, Homo sapiens
  misc_feature: Incyte ID No 1459432R1
gggtggagattttcctttatttgattttattttggagaggaagggctagagcaaaaagatgatgccaacacaccgggcactagaatgacccctgcacatgcagaacacacggacactcaagctggattagtgactgagcaaatgtgccccgtggagagaatgtcaccagagctgcaaaagccccccgaccccagcttttattagttttaagacccccaaccacacccaccccaggtctccttgttttcagtaagcagacctcctagcaaactgggcttttactcctgtgggctcagtgccacatcccctcaaataaacatgcatcctctagagcaaaagggaaattgacaggatgctggaacgccgagagatgggatgctttatttttcattatccaccagcttgggagaaaggccaccttccatcgcaccagtgagaggcgggaaagagcgatcgggccctttcccgtctctca

SEQ ID NO: 22 - DNA, 336 bp, Homo sapiens
  misc_feature: Incyte ID No 1459432X12
gtccgggactggaacctcgctgctgcccactggtcccaacgccagcaacacctctgatggccccgataacctcacttcggcaggatcacctcctcgcacggggagcatctcctacatcgacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcgtgatccacaagctcatgggcaatggggtgtggcactttgg

SEQ ID NO: 23 - DNA, 478 bp, Homo sapiens
  misc_feature: Incyte ID No 3001554F6
gagaatgtcaccagagctgcaaaatctccccgaccccagcttttattagttttaagacccccaaccacacccaccccaggtctccttgttttcagtaagcagacctcctagcaaactgggcttttactcctgtgggctcagtgccacatcccctcaaataaacatgcatcctctagagcaaaagggagattgacaggatgctggaacgccgagagatgggatgctttatttttcattatccaccagcttgggagaaaggccaccttccatcgcaccagtgagaggcgggaaagagcgatcgggccctttcccgtctctcaggccttgtgcaacatggccctggctgctcactccagccctgcctgactttaaacaaacccagtcagtacccttccacctcttgccttgggaagaagacatttgagagctcacagatatagtgcaaccggttatccaaaccaacatgttctcttgctca

SEQ ID NO: 24 - DNA, 279 bp, Homo sapiens
  misc_feature: Incyte ID No SAAC00257R1
tccccaaagtgccncaccccattgcccatgagctggtggatcatgaagggcatgcccaggagaaagaggagatctactaccgagaggttgatgatgaagatgtcggggacgttgttgcaccagtgcagcttggacttcttcacgaccgcgaagatgaccgtggagttcccgatgatgcccaggaggcagatggtgccgaacaccgaaggcatgatgatgttgatgtaggagatgctccccgtgcgaggaggtgatcctgccgaagtgaggttatcgggg

SEQ ID NO: 25 - DNA, 519 bp, Homo sapiens
  misc_feature: Incyte ID No SAAB00250R1
ggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgcctttagtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtcca

SEQ ID NO: 26 - DNA, 535 bp, Homo sapiens
  misc_feature: Incyte ID No SAAB00523R1
ggcgggaaagagcgatcgggccctttcccgtctctcaggccttgtgcaacatggccctggctgctcactccagccctgcctgactttaaacaaacccagtcagtacccttccncctcttgccttgggaannngncatttgagagctcacagatatagtgcaaccggttatccaaaccaacatgttctcttgctcagcttctgttctatccaaaggtctcatcctgctcccccaaggggatttctgatatctgaaaaccccaaacctgactccaggcctccccagcaacgtgtgagccccatggaatgtatttatttcattgcaacaacccctcacaacccggccttcctgcatttcccgagcggtcttgggtttttctcagcatctctcccggtggcgtgttgtggtgccctgacttggaggtgtgcagggtggcaggggaagtatcaggtgccttgctttctggcctctctcgtcagccgnctgagcgttgctgacagcgcgagtgcccctgggtgcaggcttaacganagctg

SEQ ID NO: 27 - DNA, 255 bp, Homo sapiens
  misc_feature: Incyte ID No 2214673H1
cctcaccagagctctggtggccacctctgtcccgccatgctgctcaccgacagtggccagggcccacagcaccaagaggcttgggccacaaagtaaagggtcgcggacctcgccggccgccatgtggagctgcagctggttcaacggcacagggctggtggaggagctgcctgcctgccaggacctgcagctggggctgtcactgttgtcgctgctgggcctggtggtgggcgtgccagtgggcctgtgctacaa

SEQ ID NO: 28 - DNA, 363 bp, Homo sapiens
  misc_feature: Incyte ID No 3073644H1
cagcaagctccaacggctgatgaaaaagctgccctgcgggggccggcactgctccccggaccacatgggggtgcagcaggtgctggcgtaggcggcccagccctcctggggagacgtgactctggtggacgcagagcacttagttaccctggacgctccccacatccttccagaaggagacgagctgctggaagacaagcaggaggggtgtttttcttgaagtttcctttttcccacaaatgccactcttgggccaaggctgtggtccccgtggctggcatctggcttgagtctccccgaggcctgtgcgtctcccaaacacgcagctcaaggtccacatccgcaaaagcctcctcgccttca

SEQ ID NO: 29 - DNA, 281 bp, Homo sapiens
  misc_feature: Incyte ID No 3573501F6
cgcacagctgngcaggtcctcaccagagntctggtggccacctctgtccnggcatgctgctcaccgacagtngccanggcccacagcaccaanaggcttgggccacaaagtaaagggtcgcggannctcgncggccgcnatgtngagctgcagctngttcaacggcacagggctgntggagganctgcctgcctgccaggacctgcagtggggntntcactgttgtcgctgctgggcctggtggtnggcntnccagtgggcctgtgctacaacgccctgct

SEQ ID NO: 30 - DNA, 238 bp, Homo sapiens
  misc_feature: Incyte ID No 4618526H1
gcagggaggacacgcccctggaccgggacacgggccggctggagccctcggcacacaggctgctggtggccaccgtgtgcacgcagtttgggctctggacgccacactatctgatcctgctggggcacacggccatcatctcgcgagggaagcccgtggacgcacactacctggggctactgcactttgtgaaggatttctccaaactcctggccttctccagcagctttgtgacacc

SEQ ID NO: 31 - DNA, 259 bp, Homo sapiens
  misc_feature: Incyte ID No 4857037H1
tttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgtaggcggcccagccctcctggggagacgtgactctggtggacgcagagcacttagttaccctggacgctccccacatccttccagaaggagacgag

SEQ ID NO: 32 - DNA, 275 bp, Homo sapiens
  misc_feature: Incyte ID No 5025086H1
cttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgca

SEQ ID NO: 33 - DNA, 563 bp, Homo sapiens
  misc_feature: Incyte ID No 1482004T1
ttntgtttatttatattctttagttttgtgcacactttgaggaattgatttaggacaggttcatactgaaaaaaacctcagctgatgttatctgtgngngctggggagggtgtcagggacatttggtggctgaggagagcgcgtcactgctattgaatagctccatttaacaccagccatgtctccgcgtctcaggcacttctgtgaaatgttctcagaaccctgtggtgactgcggcacacccggcaggccttgctagcacacgccgcccactggcagggcccggccaccctggctgttgccattctttcgtagggttttgttcattttactatttgtcatttttctaggaaacatctgtttttgtaaaacaaacaagggggaatcaagtattttaaccacaaagtataaatactggctctaagctttcatcacttcattgacaaactgaatgctgaggaggctgaaggcgaggaggcttttgcggatgtggaccttgagctgcgtgtttgggagacgcacaggcctcggggagactcaagccagatgccagccacggggct

SEQ ID NO: 34 - DNA, 466 bp, Homo sapiens
  misc_feature: Incyte ID No 153210R6
gtcatttgcatgcnaccttatatcaggtctgagaacaagctgtatgccatgtcaatcatttctctctgctgtgccgactgcttaatgggaatatatttattcgtgatcggaggctttgacctaaagtttcgtggagaatacaataagcatgcgcantgtggatggagagtactcattgtcagcttgtaggatctttggccattctgtccacagaagtatcagttttactgttaacatttctgacattggaaaaatacatctgcattgtctatccttntagatgtgtgagacctggaaaatgcagaacaattacagttctgattctcatttggattactggttttatagtggtttcattccattgagcaataaggaatttttcaaaaactactatggcaccaatggagtatgcttccctcttcattcagaagatacagaaagtattggagcccagatttattcagtg

SEQ ID NO: 35 - DNA, 230 bp, Homo sapiens
  misc_feature: Incyte ID No 2488822H1
ctttgacctaaagtttcgtggagaatacaataagcatgcgcantgtggatggagagtactcattgtcagcttgtaggatctttggccattctgtccacagaagtatcagttttactgttaacatttctgacattggaaaaatacatctgcattgtctatccttttagatgtgtgagacctggaaaatgcagaacaattacagttctgattctcatttggattactggttt

SEQ ID NO: 36 - DNA, 483 bp, Homo sapiens
  misc_feature: Incyte ID No 3558664T6
tcttgctgatgcacatgacaggtaaagctctactttaaactaggaactgcagatggactttgtatagtcttttgtcattaaacaccatctacagattgaaaggttctgcactgtctacttccaggactatattgcaatgctatgcacatagnnnnnnnnnnnnnnnnnnnnnnnnnnnnnnnnnnnnnnnnnngttactgaagtagatttctcttaatttcttatgcaaaatgtctactaatatatatacattattgatataattacttccctttgtaagagcattagtcatttttatttttcctcatgtccttgtaaaatatttatcttagcnattattataaattaattttgtggtattcatttcataccagtaaatccctcatgaagcacccccacagtattctctgcgaagaaatgaatttcagagtcagtcatgaatagganttgagtctcgttgattgaggaatcagtgacatttca

SEQ ID NO: 37 - DNA, 612 bp, Homo sapiens
  misc_feature: Incyte ID No 2488822X308B1
ggggtatgtgaaaaggtccggctccattaactcaggtggcatctcctgcagtggccacatttccacccagatgaatgatggagcatatgttttctgacctttgctgtccatagattttctttgtctgtagttataccaaaaccgatgaatcatttctttaaatggtcttgtggtcagagtatagagaattgggttcaaagcactgttaatgggcagaataaaaatcactacccaagaggttatggtacctggtatttctacctgaagcagtgaaagaaatttcactacaaaaatgggtatccagcataatgcatcagtaaatactataaagaaaaaacgtttggcaaggatcatctcttttttaacttgattccgtatttcagttgctgttatggcactttgatgaacactataaaacatgcttccataggaaaaaactgtgatgataaatgcggccaaattaataccaagaaaaattgccactgaataaatctggggctccaatactttctgtatcttctgaatgaagagggaagcatactccattggtgccatagtagntttgaaaaattccttattgctcaatggaatgaaagccacttttaaaccagt

SEQ ID NO: 38 - DNA, 562 bp, Homo sapiens
  misc_feature: Incyte ID No 2488822X310D1
agagtaagtgttaactaaaagcattttattaaattgtccttcacagaaactcaatttattaaaccatgtataatacatgttcctttgattgattattaatttgatatttttagcagcctagaagggattgaaatttcaaatatccaacaaaggatgtttagacctcttatgaatctctctcacatatattttaagaaattccagtactgtgggtatgcaccacatgttcgcagctgtaaaccaaacactgatggaatttcatctctagagaatctcttggcaagcattattcagagagtatttgtctgggntgtatctgcagttacctgctttggaaacatttttgtcatttgcatgcnaccttatatcaggtctgagaacaagctgtatgccatgtcaatcatttctctctgctgtgccgactgcttaatggggatatatttatncgtgatcngaggctttgacctaaagtttcgtggagaatacaataagcatgcgcctgtgggatngagagtactcattgtcagcttgtaggatctttggccattcctgtccncaggag

SEQ ID NO: 39 - DNA, 260 bp, Homo sapiens
  misc_feature: Incyte ID No 2705201H1
accatctgctctgtccgccgcttcctctggggcgtcctctttgcgctctgcttctcctgcctgctgagccaggcatggcgcgtgcggaggctggtgcggcatggcacgggccccgcgggctggcagctggtgggcctggcgctgtgcctgatgctggtgcaagtcatcatcgctgtggagtggctggtgctcaccgtgctgcgtgacacaaggccagcctgcgcctacgagcccatggactttgtgatggccctcatcta

SEQ ID NO: 40 - DNA, 264 bp, Homo sapiens
  misc_feature: Incyte ID No 3141184H1
cttccacgccatccctgagatccactgcacccttctgccagccctgcaggagaacacgcccaactacttcgacacgtcgcagcccaggatgcgggagacggccttcgaggaggacgtgcagctgccgcgggcctatatggagaacaaggccttctccatggatgaacacaatgcagctctccgaacagcaggatttcccaacggcagcttgggaaaaagacccagtggcagcttggggaaaagacccagcgctccgtttagaag

SEQ ID NO: 41 - DNA, 505 bp, Homo sapiens
  misc_feature: Incyte ID No 384797R6
cgtgcagctgccgcgggcctatatggagaacaaggccttctccatggatgaacacaatgcagctctccgaacagcaggatttcccaacggcagcttgggaaaaagacccagtggcagcttggggaaaagacccagcgctccgtttagaagcaacgtgtatcagccaactgagatggccgtcgtgctcaacggtgggaccatcccaactgctccgccaagtcacacaggaagacacctttggtgaaagactttaagttccagagaatcagaatttctcttaccgatttgcctccctggctgtgtctttcttgagggagaaatcggtaacagttgccgaaccaggccgcctcacagccaggaaatttggaaatcctagccaaggggatttcgtgtaaatgtgaacactgacgaactgaaaagctaacaccgactnccgcccctcccctgccacacacacagacacgtaatacagaccaacctcaatcccgcaattcganggggggcc

SEQ ID NO: 42 - DNA, 606 bp, Homo sapiens
  misc_feature: Incyte ID No 2705201X325F1
gtaggctggtgcggcatggcacgggccccgcgggctggcanctggtgggcctggcgctgtgcctgatgctggtgcaagtcatcatcctgtggagtggctggtgctcaccgtnctgcgtgacacaangccagcctncgcctacgagcccatggactttgtgatggccctcatctacgacatggtactgcttgtggtcaccctggggctggccctcttcactctgtgcggcaanttnaagaggtggaagcttaacggggcttcctcctcatcacagccttcctctctgtgctcatctgggtggcctggatgaccatgtacnttttcggnantttnaacctgcanagggggancntttnaannaccccacttggctannaantttgncggnaanngntgggttttnannatcttccatgcntccttganaccaatgcacnttttgccaacccttanggagaacannccaaactacttngaanntcccnncccatgttngggananggccttcncaggaggaattttatcttncgcggggctaanttgnnaanaaggcttncncantgnttnaannaattnagcttnccgaanncagggntttccaaacg

SEQ ID NO: 43 - DNA, 655 bp, Homo sapiens
  misc_feature: Incyte ID No 1262948X325F1
gaacagncttggagcgtcggcgctgcggggccgcgggggtcgaatgttcgtggcatcagagagaaagatgagagctcaccaggtgctcaccttcctcctgctcttcgtgatcacctcggtggcctctgaaaacgccagcacatcccgaggctgtgggctggacctcctccctcagtacgtgtccctgtgcgacctggacgccatctggggcattgtggtngaggcggtggccggggcgggcgccctgatcacactgctcctgatgctcatcctcctggtgcggctgcccttcaaggagaaggagaagaanggccctgtgngctccactttctgttcctcctggggaacctggggcctctttggggctgacgtttccttcatcatccaggaagacgagaccaatctgctnctgttccggcngcttcctcttggggggttctcttttnggctcttgctttcttcctgcctncttangcaaggcaatngcnccnttcngaagcttggttccggcantggcanggggccccccnggnttgtcaaacttnttgggcttgncgcctnttccctnaaagcttggtcaaaataatnatnccntttgaanttgcttggtntcnacccttttttnttaaaaaaaggcnaactttgcncttaaaaa

SEQ ID NO: 44 - DNA, 207 bp, Homo sapiens
  misc_feature: Incyte ID No 3036563H1
gtcacctgtatctgtgaccacctaacatcattctccatcctcatgtcccctgactccccagatcctagttctctcctgggaatactcctggatattatttcttatgttggggtgggcttttccatcttgagcttggcagcctgtctagttgtggaagctgtggtgtggaaatcggtgaccaagaatcggacttcttatatgcgccac

SEQ ID NO: 45 - DNA, 264 bp, Homo sapiens
  misc_feature: Incyte ID No 4457161H1
atcttgatggagcagaatcagtactgacagtcaagacctcgaccagggagtggaatggaacctatcactgcatatttagatataagaattcatacagtattgcaaccaaagacgtcattgttcacccgctgcctctaaagctgaacatcatggttgatcctttggaagctactgtttcatgcagtggttcccatcacatcaagtgctgcatagaggaggatggagactacaaagttactttccatatgggttcctcatcccttc

SEQ ID NO: 46 - DNA, 408 bp, Homo sapiens
  misc_feature: Incyte ID No SZAH00352F1
ctcgagggtgttcaaaaactgttgatgtgtgttgtcactttaccaatgctgctaataattcagtctggagcccatctatgaagctgaatctggttcctggggaaaacatcacatgccaggatcccgtaataggtgtcggagagccggggaaagtcatccagaagctatgccggttctcaaacgttcccagcagccctgagagtcccattggcgggaccatcacttacaaatgtgtaggctcccagtgggaggagaagagaaatgactgcatctctgccccaataaacagtctgctccagatggctaaggctttgatcaagagcccctctcaggatgagatgctccctacatacctgaaggatctttctattagcataggccaagcggaacatgaaatcagctcttctc

SEQ ID NO: 47 - DNA, 413 bp, Homo sapiens
  misc_feature: Incyte ID No SZAH02656F1
ctcgagggtgttcaaaaactgttgatgtgtgttgtcactttaccaatgctgctaataattcagtctggagcccatctatgaagctgaatctggttcctggggaaaacatcacatgccaggatcccgtaataggtgtcggagagccggggaaagtcatccagaagctatgccggttctcaaacgttcccagcagccctgagagtcccattggcgggaccatcacttacaaatgtgtaggctcccagtgggaggagaagagaaatgactgcatctctgccccaataaacagtctgctccagatggctaaggctttgatcaagagcccctctcaggatgagatgctccctacatacctgaaggatctttctattagcataggcaaagcggaacatgaaatcagctcttctcctggg

SEQ ID NO: 48 - DNA, 489 bp, Homo sapiens
  misc_feature: Incyte ID No SZAH01730F1
ccctccattgtccttctcccaaactaatgtgcagatgagcagcatggtaatcaagtccagccacccagaaacctatcaacagaggtttgttttcccatactttgacctctggggcaatgtggtcattgacaagagctacctagaaaacttgcagtcggattcgtctattgtcaccatggctttcccaactctccaagccatccttgctcaggatatccaggaaaataactttgcagagagcttagtgatgacaaccactgtcagccacaatacgactatgccattcaggatttcaatgacttttaagaacaatagcccttcaggcggcgaaacgaagtgtngtcttctggaacttcaggcttgccaacaacacaggggggtgggacagcagtnggtgctatgttgaagaaggtgatggggacaatgtcacctgtatctgtgaccacctaacatcattctccatcctcatgtcccctgacttcccagatc

SEQ ID NO: 49 - DNA, 87 bp, Homo sapiens
  misc_feature: Incyte ID No SZAH03622F1
ccaagacagaaggcaatggctttctgagtggacctgcttgtttcatgcagaatgaaaaccaaggggtagaacagcattagggccaat

SEQ ID NO: 50 - DNA, 116 bp, Homo sapiens
  misc_feature: Incyte ID No SZAH01163F1
cttctgttcccgtgtggtcacgtaggttggattgtcctgttcttagttgtgcaacgaagaatgctcttggatgagttttccagggatgatctggtttcttctgtgttggaatcgtg

SEQ ID NO: 51 - DNA, 558 bp, Homo sapiens
  misc_feature: Incyte ID No SZAH02669F1
cactgtccccgggaggtcacgtaggttggattatcctgttcttagttgagcaacgaagaagcactggatgagttttccagggatgagctggttgcttctggggtggaaacattatacgttcctgttttaccaaacaaattgttaaatctccttgatattggagaactcatagaaaacacaggtgtggatgaacccagggatgtcgactttgagtgctgtgaagaccatctcgacaatgaaaacttattcagcaaagcttcctgtaccttcagatcccagaggcatccaaagagtaaaatgaataatccctggaagacattgaggatggcaaatatgatatggaacacaaggttggtccctgggaacacagtggtgagaccaaaaccccaagtgaggcccaagagtggtgtgaggaccccaatgctcttgctgatctgaaacaggctgctcttctcctgcttgcatggcttgtctccaatggaaggcctcaggatcttggtgatgacacaatagtgatggttatgttcaccacacaatgatcagtgctgggatggcaaa

SEQ ID NO: 52 - DNA, 362 bp, Homo sapiens
  misc_feature: Incyte ID No SZAH00249F1
ctcatccctggaaaactcatccagtgcttcttcgttgctcaactaagaacaggataatccaacctacgtgacctcccggggacagtggctgtgcttttaaaaagagatgcttgcaaacaatggggaacgtgttctcggggcaggtttccgggagcagatgccaaaaagactttttcatagagaaggggctttcttttgtaaagacagaataaaaataattgttatgtttctgtttgttccctccccctcccccttgtgtgataccacatgtgtatagtatttaagtgaaactcaagccctcaaggcccaacttctctgtctatatgtaatatagatttccgagaggcattttcaccttttac

SEQ ID NO: 53 - DNA, 615 bp, Canis familiaris
  misc_feature: Incyte ID No 702778992H2
cggggccttcgtgctgctcaccacggccacctccattgccatatgggtggtgtggattgtcatgtacacgtacggcaacaggcagcgcaacagccccacctgggatgaccccacgctggccatcgccctcgccgccaatgcctgggcctttgtgctcttctatgtcatccctgaggtctcccaggtgaccaaggccagcccagagcaaagttaccagggggacatgtaccccacccggggcgtaggctacgagaccatcctgaaagagcagaagggccagagtatgtttgtggagaacaaggcattttccatggatgagccagcctcagctaagagaccggtgtcaccatacagtgggtacaacgggcagctgctgaccagcgtgctccagcccaccgagatggccctgatgcacaaaggcccgtccgaaggagcttacgacgtcatcctcccacgagccaccgccaacagccaggtgatgggcagtgccaactccaccctgagggccgaagacatggttgcggcccagagccaccaggcagccacgccaccgagagacggcaagagctcccaggtctttagaaacccctacgtgtgggactgagtcggcggcag

SEQ ID NO: 54 - DNA, 686 bp, Rattus norvegicus
  misc_feature: Incyte ID No 701938522F6
accacggccacctccattgccatctgggtggtgtggattgtcatgtacacctacggcaataagcagcaccatagccccacctgggatgaccccacactggccattgcgctcgctgccaatgcctggacttttgtcttcttctatgtcatccctgaggtctcccaagtgaccaaacccagcccagaacagagctaccagggggacatgtacccgacccgaggggtgggctacgagaccatcctgaaggagcagacgggccagagcatgttgtggagaacaaggcattttctatggatgaaccagcctcagcaaagagaccggtgtcgccttacagtggctacaatggtcagctgctgaccagcgtgtaccagcccaccgagatggccctgatgcacaaaggcccgtctgaaggtgcgtacgacgtcatcctcccacgggccaccgcaacagccaggtgatgggcagtgccaactcaaccctgcgagctgaagacatgtacatggtccagagccaccaggtggcacgccaacgaaagacggcaagatctctcaggatcagtccccgaaaaataaaacaagatggtagatgccctcttccctggaccgtgacctctccgtgtgccattgccaacatggactttgtcatggcctcatttacgtaatgctgctgctgctggcggcttccta

SEQ ID NO: 55 - DNA, 198 bp, Macaca fascicularis
  misc_feature: Incyte ID No 700712581H1
tggcttgccgcgcggcagcggctgccaggctgcccgccgaagacccccttcccgactgcggggcttgggctcctggacaaggtggcaggtgctggaggctgccgcagtctgcgtgggtggaggggagctcagcttggttgtgggagccggcgaccgtcactggctggatggacctggaagcctcgctgctgcccactg

SEQ ID NO: 56 - DNA, 271 bp, Mus musculus
  misc_feature: Incyte ID No 701250242H1
aagaaatccaagctgcactggtgcagcaacgtccctgacatcttcatcatcaacctctctgtggtggatctgcttttcctgctgggcatgcctttcatgatccaccagctcatgggtaatggtgtctggcactttggggaaaccatgtgcaccctcatcacagccatggacgccaacagtcagttcaccagcacctacatcctgactgctatggccattgaccgctacttggccaccgtccatcccatctcctccaccaagttccggaagc

SEQ ID NO: 57 - DNA, 304 bp, Rattus norvegicus
  misc_feature: Incyte ID No 701899983H1
ccaccccatctcctccaccaagttccggaagccctccatggccaccctggtgatctgcctcctgtgggcgctctccttcatcagtatcacccctgtgtggctctacgccaggctcattcccttcccagggggtgctgtgggctgtggcatccgcctgccaaacccggacactgacctctactggttcactctgtaccagtttttcctggcctttgcccttccgtttgtggtcattaccgccgcatacgtgaaaatactacagcgcatgacgtcttcggtggctccagcctcccaacgcagcatc

SEQ ID NO: 58 - DNA, 248 bp, Rattus norvegicus
  misc_feature: Incyte ID No 701028051H1
ggcgacctgcaccggctgcatggatctgcgaacctcgttgctgtccactggccccaatgccagcagcatctccgatggccaggataatctcacattgccggggtcacctcctcgcacagggagtgtctcctacatcacatcattatgccttccgtgtctggtaccatctgtctcctgggcatcgtgggaaactccacggtcatctttgctgtcgtgaagaagtccaagctacactggtgcagcaacgt

SEQ ID NO: 59 - DNA, 497 bp, Mus musculus
  misc_feature: Incyte ID No 075474_Mm.1
gtgacactgctcatcctgttcaacgtggcttccctggtgaccatgtactccactgcactgctgagccttgactactacatcgagcgtgccctgccaccacctacatggccagtgtgtacaacacccggcacgtgtgtggcttcgtctggggaggggcggtgctcaccagcttctcctccctgctcttctacatctgcagtcacgtgtcttctagaatcgctgagtgtgcccggatgcagaacacggaggcagccgatgctatccttgtgctcatcggctacgtggtgccaggtctggctgtgttgtatgccctggcactcatctcgagaatcgggaaggaagacacacccctggaccaggacaccagcaggctggacccctcggtgcacaggctgctggtggccaccgtgtgcactcagtttggcctctggacaccttactacttgagcctggggacacagtgctgacgtcacgggggaggaccgtggaggggcatt

SEQ ID NO: 60 - DNA, 266 bp, Rattus norvegicus
  misc_feature: Incyte ID No 700819903H1
gtgtgtacaacacccggcacgtgtgtggcttcgtctggggaggggcagtgctcaccagcttttcctccctgctcttctatatctgcagtcatgtgtcttctagaattgccgagtgtgcccggatgcagaacacggaggcagccgacgccatccttgtgctcattggctacgtggtgccaggtctggctgtgttgtatgccctggcactcatctcaaggattgggaaggaagacacacccctggaccaggacaccagcaggctggac

SEQ ID NO: 61 - DNA, 294 bp, Rattus norvegicus
  misc_feature: Incyte ID No 701657796H1
ggaagacacacccctggaccaggacaccagcaggctggacccctcagtgcacaggctgctggtggccactgtgtgcacacagtttggcctctggacaccttactacctgagcctggggcacacagtgctagtgtcacggggaaggaccgtagtggggcattatctgggcatcctacaggttgctaaggacctggcgaagttcttggccttctcaagcagttctgtgacgccgctgctctaccgttacatcaacaaagccttccccagcaagctccggcgcctggtgaagaagat

SEQ ID NO: 62 - DNA, 432 bp, Rattus norvegicus
  misc_feature: Incyte ID No 702466096T1
aatgggaatccagcacaattgctatcggttgaacacaataaagaaaaagcgtttggcgaggatcatctccttcttcacctgcttctgtatttcggtggctgttatggtgctttgatgaacactgtaaaacatgcttccataggagaacacaatgatgataaacgccaccaggtttaatacctgtttagaccatgaagaatattagtagtgtatgctagcattctcttaagacaaacatggcttagatgtcactattaaagatcacagagcccataaagtggtattcatttattcgtttatttactctgtgacaaggtcttattgtagagttcagatgagccttcaacttgactaggtagcctaggctggacaccaacatgcagtcctcctgcctcagattacaaatgtgtaccagatcttcctgatctccat

SEQ ID NO: 63 - DNA, 727 bp, Macaca fascicularis
  misc_feature: Incyte ID No 703021534H1
gagggccagccccagggtgaccaccagcagtaccatgtcgtagatgagggccatcacaaagtccatgggctcataggcgcaggccggcctcgtgtcgcgcagcacggtgagcaccagccactccacagcgatgatgacttgtaccagcatcaggcacagcgccaggcccaccagctgccagcccgcggggcccgtgccgtgccgcaccagcctccgcacgcgccacgcctggctcagcaggcaggagaagcagagcgcaaagaggacgccccagaggaagcggcggacggagcagatggtctcgtcctcctggatgatgaaggcgaatgtcagcccgaagaggcccagggtccccaggaggaagagaaagtggaggcccacggggctcttcttctccttctccttgatgaagggcagccgcaccaggaggatgagcatcaggagcagtgtgatcagggcgcccgccccggccaacggcttcaacaagaagtgccccagatggcgtccaggtcgcacagggacacgttactgagggacggcaggtccagcccgcaccctcgggacgtgctggcgttttcagaggccaccgaggtgatcacaaagagcaggaggaaggtgagcacctggtgagctctcatctttctctctgatgccacgaacattcgacccctgcggcccgcagcgccaacgctccagctgggcctcggcccgagtcacatctctgcag

SEQ ID NO: 64 - DNA, 461 bp, Canis familiaris
  misc_feature: Incyte ID No 703543565J1
cagagggacaggagggcagtcggtgttagcttttcggttcagcagtgttcacatttacacgaaatccccttgtgtaggatttctagatctcccggctgtgaggcagccttgttcggctactgttactgatttctccctcaagaaagacacagccagggaataaaatcggtaacgagagattcttacttctctggaacttaacacagtctttcaccagaggtgtcttccagtgctaactaggcggagcagttgggatagtccctccatcgagcacaacggccatctcagctgggctgactagacacttgctctctaaacggagcgctcggtctgtttcccaagctgccattgcgacaatcccgccgttcggagagctgcatagtgttcatccatcgagaaggcttcgcttctccatgtaggtccgtggcagctgcacgtcctcctcacaacgcatgtctccc

SEQ ID NO: 65 - DNA, 278 bp, Mus musculus
  misc_feature: Incyte ID No 076599_Mm.1
cgcgggcgcgctgcagagatgtgacttgggcccagggccagcaggagcgtcggcgctgcggggacgcgagggtcgaatgttcctggtgttagagagaaagatgagaacccatcaagtgtttcccttgcccctgctcctggtgattgcctccgtggcttcagagaacgccagcacgtcccggggctgtggactggaccttcttcctcagtacgtgtccctgtgcgacctggacgccatctggggcatccntggtggagggcagtggccggggcgggggc

SEQ ID NO: 66 - DNA, 561 bp, Rattus norvegicus
  misc_feature: Incyte ID No 701749639H1
gaggcggctgtgtgcctccacttcctcttcctgctggggaccctgggcctctttggcctgacgtttgctttcatcatccggatggacgagacaatctgctccatccgacgcttcctctggggtgtcctcttcgcactctgcttttcctgcctgctgagccaggcgtggcgggtacggaggctggtgcgccagggcacgagcccggccagctggcagctggtgagcctggcactgtgcctgatgctggtgcaggtcatcatcgccactgagtggctggtgctgactgtgctacgtgacacgaagccggcctgcgcctacgagcccatggattttgtgatggcgctcatctacgacatggtgctgctggctatcaccctagcgcagtccctcttcacactgtgtggcaagttcaagcggtggaaggtgaacggagccttcatcctcatcactaccttcctctctgtgctcatctgggtgatctggatgaccatgtacctcttcggcaactcgttaattaagcgggcagatgcctggagcgaacctaccttggccatcacgctg

SEQ ID NO: 67 - DNA, 499 bp, Rattus norvegicus
  misc_feature: Incyte ID No 702147192H1
gcgctgcggggacgcgagggtcgagtgttcctggtgtcagagagaaagatgagaacccaccaagtgcttcccttgcccctgctcctggtgattgcctctgtggcttcggagaacgccagcacgtcccggggctgtgggctggaccttcttcctcagtacgtgtccctgtgcgacctggacgccatttggggaatcgtggtggaggcagtggccggggcaggggccctgatcacactgcttctgatgcttattctcctggtgagactgcccttcatcaaggacaaggaaaggaggcggcctgtgtgcctccacttcctcttcctgctggggaccctgggcctctttggcctgacgtttgctttcatcatccggatggacgagacaatctgctccatccgacgcttcctctggggtgtcctcttcgcactctgcttttcctgcctgctgagccaggcgtggcgggtacggaggctggtgcgccagggcacgagcccggcca

SEQ ID NO: 68 - DNA, 565 bp, Canis familiaris
  misc_feature: Incyte ID No 703557532J1
gctgttcagatcagcaagagcatnggggtcctaacaccactctggggctcacctggggtttggtcttgccactgtgttccaaggaagcaagctgtgttccatattatatttacactcctcaatgcctttcagggattattcatttgctcttggatgcctctgggatcagaaggtacaggaagccttactaaagaagttttcactgtcaagatggtcttctcagcactcaaagtcaacatccctaggttcatctacaccagtattttctatgagttctccaatatcaagaagatttaacaatttattggaaaaacaggaacgtacaagtttccaccccagaaacaaccagctcatccctggaaaacacatccagtgcttactccttgctgaactaagaacaggaaaatctacccacgtgacttcttaaaggacagcggatatgctctgaaaaaaaaaaaaaatcctttcaaagccatggggtaaaacggtttcctccgaggcttcccgggagcaaatgctgaagagacctttcggctttaggggaaaagaagcttcctttggtaaa

SEQ ID NO: 69 - DNA, 468 bp, Canis familiaris
  misc_feature: Incyte ID No 702766139H1
cccgccagtaggactccagagatgtttggtacttttgagaaatggcagagtttctggatgacttttccaggctccccaacacctattacgggatctcggcacatgatgttctttccaggaaccacattaagcttcatagatgggctccggactgaattattagcagcattaggtaaagtgacaaaatatgtccagcttttttagacaccaggaaactgatgtccttgccatgaacttgtatttgcagcacacttgcttgccattaacttctttttctgcaggaaaggataaggaatccacttggaaagtcactctgtagtatctcagtcctcgtcaatgcagcatctgaagtgatagggaacccttgcagggaactgtagcactccagaggatcaaccatgatgtttggctctagaggcagtgggtaaacggtcacatctttcattacgacacatgtatgaatacttg

SEQ ID NO: 70 - DNA, 263 bp, Mus musculus
  misc_feature: Incyte ID No 701085654H2
ctattccagatcagcaagagtatcggggtcctcacaccactcttggggctcacttggggtttcggtcttgccacagtgatccagggaagcaatgctgtgttccacatcatatttactactctcaatgccttccaggggctcttcattttgctctttggctgcctctgggatcagaaggtgcaggaagctttgctgcataagttttcattgtcaaggtggtcttctcaacactcaaagtcaacatccataggttcgtcaacacc

SEQ ID NO: 71 - DNA, 246 bp, Mus musculus
  misc_feature: Incyte ID No 701077530H1
cctcattatctcctctatcacagtgggggttacgcagctacaggaagtctacatgatgaagaacgcgtgttggctcaactgggaggacaccagagcactgctggcttttgccatccccgcgttgattattgtggtggtaaatgtgagcatcacagttgtggtcatcaccaagatcctgaggccctccattggggacaagccaggcaagcaagagaagagcagcctattccacatcagcaagagtat

SEQ ID NO: 72 - DNA, 515 bp, Rattus norvegicus
  misc_feature: Incyte ID No 702147631H1
gttgtggaagccatggtgtggaaatcagtgaccaagaaccgaacttcctatatgcgccacatctgcatcgtcaacattgccttttctctaggctatggctgtccactcattatctcatccatcacagtgggggttacacagccacaggaagtttacatgaggaagaatgcatgttggctcaactgggaggacaccagagcactgctggcttttgctatcccagcgttgattattgtggtggtgaacgtgagcatcacagttgtggtcatcaccaagatcctaaggccctccgtcggagacaagccaggcaagcaggaaaagagcagcctattccagatcagcaagagcattggagtcctcacgccactcttggggctcacttggggttttggtctggccacagtgatccaggggagcaatgctgtgttccacatcatatttactctcctcaatgcctttcaggggctcttcattttgctctttggctgcctctgggatcagaaggtacaggaagc

SEQ ID NO: 73 - DNA, 539 bp, Rattus norvegicus
  misc_feature: Incyte ID No 702239655H1
ggatatcatttcttacatcgggttgggcttttccatagtcagcttagctgcctgtctagttgtggaagccatggtgtggaaatcagtgaccaagaaccgaacttcctatatgcgccacatctgcatcgtcaacattgccctttgccttctgattgctgacatctggttcattgtggctggtgctatccatgatgggcattacccactcaacgaaacagcctgtgtggccgccacattcttcattcacttcttctacctcagtgtcttcttctggatgctaactctgggcctcatgctcttctaccggctgattttcattctacatgacgcgagcaagtccacgcagaaagccattgccttttctctaggctatggctgtccactcattatctcatccatcacagtgggggttacacagccacaggaagtttacatgaggaagaatgcatgttggctcaactgggaggacaccagagcactgctggcttttgccatcccagcgttgattattgtggtggtgaacgtgagcatcacacaca

SEQ ID NO: 74 - DNA, 571 bp, Rattus norvegicus
  misc_feature: Incyte ID No 702438348T1
tctgtctttacaaaagaaagcatcttctctattcaaagagtctcttcagcatctgctcccagaagtctgcagagagaacactttacccatagatttggatatgggtcccttttcttggcaggggccctatttctgagagctcctgtgaatttggcattatctggtcctagttgagcaatgagtaagcactagaggaattttccacggatgagctggttgtctctggggtggaaacgttatatgttccatcaggaggatgaactgccactgataacaaggtgtccatcattgccttgggggacctttggggctgctgttttaccaaaaagattattaaatcttcgggatatcggagaactcatcgaaaacacaggtgttgatgaacctaaggatgttgactttgagtgttgagaagaccaccttgacaatgaaaacttatgcagcaaagcttcctgtaccttctgatcccagaggcagccaaagagcaaaatgaagagcccctgaaaggcattgaggagagtaaatatgatgtggaacacagcattgctcccctggatcactgtggccagac